(19) United States
(12) Patent Application Publication
(10) Pub. No.: US 2007/0275408 A1
(43) Pub. Date: Nov. 29, 2007

(54) Synthetic Bioelastomer

(76) Inventor: Christopher Malcolm Elvin, St. Lucia (AU)

Correspondence Address: SUGHRUE MION, PLLC, 2100 PENNSYLVANIA AVENUE, N.W., SUITE 800, WASHINGTON, DC, 20037, US

(21) Appl. No.: 10/557,445

(22) PCT Filed: May 21, 2004
(86) PCT No.: PCT/AU04/00683

§ 371 Date: Aug. 14, 2006

(30) Foreign Application Priority Data
May 21, 2003 (AU) 2003902483

(51) Int. Cl. C07K 14/78; B82B 1/00; C07K 19/00; B82B 3/00

(52) U.S. Cl. 435/7.1; 435/320.1; 435/325; 435/69.1; 530/325; 536/23.5; 977/768

(57) ABSTRACT

A bioelastomer which is a polypeptide comprising a plurality of repeat units in which the consensus sequence SSXXYGXP, where S is serine, X is an unspecified amino acid, Y is tyrosine, G is glycine and P is proline, is present and which is cross-linked through dityrosine bond formation, with the proviso that the bioelastomer is not resilin.

### TECHNICAL FIELD

The present invention is concerned with a synthetic bioelastomer and, more particularly, a polypeptide bioelastomer whose amino acid sequence is derived from the repeat sequences of resilin. The present invention is also concerned with nanomachines, biosensors and like apparatus, in particular, those in which the polypeptide is, for example, a part of, a spring mechanism, or “nanospring”. The invention also provides the use of the bioelastomer in macroscopic applications. Fusion proteins with other polypeptides also form a part of the invention and may be used in various of these applications, as can hybrid molecules formed in other ways.

### BACKGROUND ART

Resilin is a rubber-like protein which occurs in specialised regions of the insect cuticle and is the most efficient elastic material known. The elastic efficiency of the material is purported to be 97%; only 3% of stored energy is lost as heat. It confers long range elasticity to the cuticle and functions as both an energy store and as a damper of vibrations in insect flight systems. It is also used in the jumping mechanisms of fleas and grasshoppers.

The first description of resilin was by Weis-Fogh (1960). This was of elastic ligaments associated with the wings of the locust and elastic tendons in the flight musculature of the dragonfly. Resilin displays extraordinary elasticity (Weis-Fogh, 1960). The elastic tendon from dragonflies can be stretched to over three times its original unstrained length without breaking and it returns immediately to its original length when the strain is released. No lasting deformations are present even after the sample has been kept in the stretched condition for weeks on end (Weis-Fogh, 1961a, 1961b).

Resilin has been found in the jumping mechanism of fleas (Bennet-Clark and Lucey, 1967) and in a number of other insect structures and in some crustaceans (Andersen and Weis-Fogh, 1964). It has been found in all insects investigated and also in crustaceans (cray-fish), but appears to be absent from arachnids.

The two most outstanding properties of resilin are its elasticity and its insolubility. It is insoluble in water below 140° C. In many solvents, resilin swells considerably, especially in protein solvents such as, phenol, formamide, formic acid. Resilin also swells without going into solution in concentrated solutions of lithium thiocyanate and cupric ethylenediamine, solvents which are able to dissolve silk fibroins and cellulose. When resilin is placed in methanol, ethanol or acetone, it shrinks to a hard glassy substance as when dried in air. When placed back in water, it swells to its original size with no noticeable change in its elastic properties (Weis-Fogh, 1960).

The elastic properties of resilin are consistent with the requirements of polymer elasticity: the cross-linked molecules must be flexible and conformationally free. There are two theories to explain elastic behaviour of materials. The first is the so called “rubber theory”, which attributes rubber-like properties to a decrease in conformational entropy on deforming a network of kinetically free, random polymer molecules. The second is the theory of Urry and co-workers (Urry, 1988; Urry et al. 1995), which proposes that the elastic mechanism arises from the beta-spiral structure. Resilin and abductin behave as entropic elastomers, returning almost all of the energy stored in deformation. However, abductin has low proline content with no predicted β-turns and hence no β-spiral. The amino acid composition of resilin is more like that of elastin, with high proline, glycine and alanine content. Nevertheless, the sequences do not show similarities in alignment however and appear to be unrelated on an evolutionary basis.

An important property of resilin is the cross-linked nature of the insoluble resilin. This has been shown to be due to tyrosine cross-linking resulting in the formation of dityrosine moieties (Andersen, 1964; 1966). The precursors of resilin are probably soluble, non-cross-surface of the epidermal cells into the subcuticular space, where they are rapidly cross-linked to form a three dimensional easily deformable protein network.

U.S. Pat. No. 6,127,166 entitled, “Molluscan ligament polypeptides and genes encoding them”, describes a mollusc protein based on the repeat sequences in abductin which can be used as a novel biomaterial. The gene encoding abductin is not related to the resilin gene (<30% identity) and the formation of beta-turns is not predicted. The repeat sequence identified for abductin is GGFGGMGGGX, which does not contain tyrosine and therefore cannot cross-link through the formation of dityrosine links, as resilin does.

A polypeptide that comprises at least three beta-turn structures is described in International Publication No. WO 98/05685. The repeat sequence disclosed is based on human elastin. Elastin typically cross-links through the oxidisation and condensation of lysine side chains to produce hydrolysates which contain desmosine and isodesmosine. There is no suggestion of dityrosine cross-link formation to link the beta-turns.

International Publication No. WO 02/00686 describes a nanomachine comprising a bioelastomer having repeating peptide monomeric units which form a series of beta-turns separated by dynamic bridging segments suspended between said beta-turns. The bioelastomers described are poor in tyrosine, and there is no suggestion of tyrosine cross-linking between the chains comprising beta-turns. To the contrary, the fundamental functional unit at the nanoscale dimension is the twisted filament, formed through coupling a plurality of individual chains to a multi-functional cap—adipic acid for the double-stranded filament, the Kemp tri-acid for the triple-stranded filament and EDTA for a quadruple-stranded filament.

The present invention is based on the discovery that a recombinant polypeptide expressed from exon 1 of the resilin gene from Drosophilia melangogaster may be cross-linked by dityrosine formation and form a bioelastomer, despite only amino acids 19-322 of a 620 amino acid polypeptide being present. A consensus sequence was derived from this observation and from observations in other species, and polypeptides with repeat sequences based on same were prepared. While not wishing to be bound by theory it is proposed that a polypeptide having an amino acid sequence in accordance with the invention comprises a series of beta-turns which together form a beta-spiral, which can act as a readily deformed spring (a “nanospring”) in nanomachines and/or be cross-linked by dityrosine bond formation to form a novel bioelastomer.

According to a first aspect of the present invention there is provided a bioelastomer which is a polypeptide comprising a plurality of repeat units with the consensus sequence SXXYGXP, where S is serine, X is an amino acid, Y is tyrosine, G is glycine and P is proline, and which is cross-linked through dityrosine bond formation, with the proviso that the bioelastomer is not resilin.

In an embodiment of the invention the repeat units comprise the consensus sequence:

X₁X₂X₃X₄SX₅X₆YGX₇PX₈X₉X₁₀X₁₁

wherein:

X₁ is absent or any amino acid;

X₂ is absent or any amino acid;

X₃ is absent or any amino acid;

X₄ is P or S;

X₅ is a charged or polar amino acid;

X₆ is a charged or polar amino acid;

X₇ is A or P;

X₈ is G or A;

X₉ is absent, G or a polar amino acid;

X₁₀ is absent, G or a polar amino acid; and

X₁₁ is absent or any amino acid.

In an embodiment, X₁, if present, is G, Y, A or N, most preferably G.

In an embodiment, X₂, if present, is a basic amino acid or G, more preferably, G, L or Q and most preferably G.

In an embodiment, X₃, if present, is a basic amino acid T or P, more preferably R, K, T or P.

X₄ is typically P.

X₅ is preferably D, T or S.

X₆ is preferably S, Q or T.

X₇ is typically A.

X₈ is typically G.

X₉, if present, is preferably G, Q or S, most preferably G.

X₁₀, if present, is preferably G, S or N, most preferably G.

X₁₁, if present, is preferably G, Q, P, S or N.

In an embodiment at least one of X₈, X₉ and X₁₀ is G, most preferably X₈.

The repeat sequences may be the same or different, and at least some may include variations from the above sequence.

The repeat sequences may be joined by linker sequences which typically comprise 1 to 20 amino acids, more typically 1 to 10 amino acids and generally 1 to 4 amino acids. Linker sequences may be absent, with the repeats abutting, or there may even be overlap of repeat units in accordance with the above sequence.

The length of the repeat sequences may vary, and it will be appreciated this will result in different spacing between the tyrosine residues. Therefore, there will be greater or lesser spacing between the dityrosine cross-links and, while not wishing to bound by theory, it is proposed that this will affect the “tightness” of tyrosine residues are separated by 8 to 34 amino acid residues, more often 9 to 24, and most often 10 to 14 residues, given the usual length of the repeat sequences and linker sequences.

There may be additional tyrosine residues within the repeat sequences, within the linker sequences or within other regions of the cross-linked polypeptide.

There may be any number of repeats, preferably from 1 to 50 repeats, more preferably from 6 to 40, more preferably still from 8 to 30, more preferably still from 10 to 25 and generally from 12 to 20.

In an embodiment of the invention the polypeptide comprises repeat units with the sequence GGRPSDSYGAPGGGN or GGRPSDTYGAPGGGN, for example, the polypeptide RPSDTYGAPGGGNGGRPSDTYGAPG. The polypeptide may comprise the amino acids shown in italics in FIG. 6, which are from exon 1 of the resilin gene (SEQ ID NO:1). The region amplified in this embodiment is not the entire exon 1. Exon 1 extends from base 54752-55771, but the region expressed in this embodiment comprises bases 54805-55714. This region excludes the putative signal sequence and includes an initiating methionine and six-histidine residues, followed by the sequence PEPPVNS . . . SDSYGPPASG and 18 repeat units. The polypeptide may be a fragment of SEQ ID NO:1, or could have the amino acid sequence set forth in SEQ ID NO:3, SEQ ID NO:7, SEQ ID NO:14 or SEQ ID NO:27. The polypeptide may be encoded, for example, by a portion of SEQ ID NO:2, by SEQ ID NO:26 or by any sequence which codes for the polypeptides, and may also be prepared by chemical synthesis.

In a further embodiment the polypeptide comprises repeat units with the sequence PSSQYGAPAQT. This shorter repeat sequence may comprise a fragment from SEQ ID NO:4 containing the repeat units or any of SEQ ID Nos:15, 17, 25, 29 or 31, or the polypeptide encoded by the DNA sequences set forth in SEQ ID Nos:17-24.

In a still further embodiment the polypeptide comprises repeat units with the sequence SSSYGAP or SSTYGAP or STTYGAP.

As used herein, the term “consensus sequence” refers to an amino acid (or nucleotide) sequence comprising the amino acids most commonly found at each portion in the sequence but may leave the nature or identity of some-amino acids unstated in which case the symbol “X” is used in place of the conventional 1 letter amino acid codes. The conventional 1 letter amino acid codes are used throughout the specification, and will be well understood by the person skilled in the art. It will be appreciated that not all repeat units will conform in their entirety to the consensus sequence and yet the beta-spiral structure of the polypeptides of the invention is not disrupted, but most or all repeat units will have the specific sequences set forth above.

According to a second aspect of the present invention there is provided an isolated polypeptide comprising a plurality of repeat units with the consensus sequence SXXYGXP, where S is serine, X is an amino acid, Y is tyrosine, G is glycine and P is proline, with the proviso that the polypeptide is not pro-resilin.

Embodiments of the isolated polypeptide are described above.

In an embodiment the polypeptide has the amino acid sequence set forth in SEQ ID Nos: 25, 27, 29 or 31.

According to a third aspect of the present invention there is provided an isolated nucleic acid which encodes a polypeptide according to the second aspect.

In an embodiment the nucleic acid has the nucleotide sequence set forth in any one of SEQ ID Nos:17-24, 26, 28 or 30.

According to a fourth aspect of the present invention there is provided a method of preparing a bioelastomer comprising the steps of:
(1) providing a polypeptide according to the second aspect;
(2) initiating a cross-linking reaction; and
(3) isolating the bioelastomer.

According to a fifth aspect of the present invention there is provided a hybrid molecule comprising a polypeptide according to the second aspect and a second polymeric molecule.

Preferred forms of the isolated polypeptide are as described above.

The following sequences may be added to the core recombinant protein sequence, producing hybrid molecules:

A. Mussel byssus protein sequences

B. Spider silk protein

C. Collagen

D. Elastin

E. Fibronectin

F. Glutenin

These hybrid polymers will display new properties including resilience with high tensile strength, adhesion properties and cell interaction and adhesion.

A recombinant form of spider dragline silk protein has been successfully expressed in transformed mammalian cells in culture (Lazaris et al. 2002).

Glutenin proteins, specifically the HMW-GS (high molecule weight glutenin subunits) are responsible for the elastic properties of dough (Parchment et al., 2001).

The mussel adhesive proteins Mefp-1,2 and 3 have also been expressed in E. coli and also synthesised chemically. (Deming, 1999)

Elastin has been produced as a recombinant form (Meyer and Chilkoti (2002).

Advantageously, the isolated polypeptide is a his-tagged polypeptide.

The polypeptides of the invention may be synthesized chemically using methods well known to the person skilled in the art. They may also be prepared by synthesis of genes encoding repetitive, protein-based polymers of specific sequence, chain length and architecture is described by Meyer and Chilkoti (2002).

For example, one might synthesise a hybrid resilin gene comprising concatamers of the resilin repeat but with variations in the number and spacing of Tyrosine residues. One might also synthesise a gene with hybrid sequences added to the resilin gene repeats. These additional genes might encode the Byssus plaque protein (Mefp) sequence or the elastin sequence or the fibronectin cell adhesion sequence motif (Arg-Gly-Asp-Ser/Val) or dragline spider silk protein sequence or collagen sequence.

These hybrid genes could then be cloned into a bacterial expression vector such as that described in the present invention for production of the novel recombinant protein(s).

Another modification includes the production of hybrid hydrogel systems assembled from water-soluble synthetic polymers and a well-defined protein-folding motif, in this case the resilin polypeptide unit. These hydrogels undergo temperature-induced collapse owing to the cooperative conformational transition of the coiled-coil protein domain. This system shows that well-characterized water-soluble synthetic polymers can be combined with well-defined folding motifs of proteins in hydrogels with engineered volume-change properties. This technology has been described by Wang et al (1999).

In an embodiment there is provided a polypeptide comprising a first polypeptide as described above and a second polypeptide fused to the first polypeptide.

The fusion protein may be cross-linked through dityrosine cross-links, but need not necessarily be cross-linked. For example, the first polypeptide comprising a series of beta-turns in sufficient number to form a beta-spiral may be fused to a second peptide without cross-linking to form a spring mechanism in a nanomachine although, the first polypeptide may be cross-linked.

Alternatively, the second polypeptide may be an enzyme, in order to allow the introduction of functionality to a bioelastomer, an immunoglobulin, a structural protein such as silk fibroin which can then be woven into an extremely light, resilient and durable thread or filament, or any other polypeptide.

According to a sixth aspect of the present invention there is provided a nanomachine comprising a polypeptide according to the second aspect acting as a spring mechanism and a device upon which said spring mechanism acts.

According to a seventh aspect of the present invention there is provided a biosensor comprising a polypeptide according to the second aspect or a bioelastomer according to the first aspect or a hybrid molecule according to the third aspect.

According to an eighth aspect of the present invention there is provided a manufactured article consisting or comprising of a bioelastomer according to the first aspect or a hybrid molecule according to the fourth aspect.

### BRIEF DESCRIPTION OF THE DRAWINGS

FIG. 1 is a schematic illustration of how elastomeric polypeptides work;

FIG. 2 shows the beta-spiral structure in UDP-N-acetylglucosamine acyltransferase;

FIG. 3 is an alternative representation of the beta-spiral elastic protein structure using a space filling model;

FIG. 4 shows the nature of the dityrosine cross-link in proteins;

FIG. 5 is a schematic illustration of cross-linking in a bioelastomer such as is created by the formation of tyrosine cross-links; resilin gene from Drosophilia melangogaster;

FIG. 7 shows the DNA sequence from the coding region of the resilin gene from Drosophilia melangogaster;

FIG. 8 shows the PCR reaction products using primers RESF3 and RESPEPR1 which shows that expression and purification of soluble Drosophilia pro-resilin in E. coli has been achieved;

FIG. 9 shows a partial NdeI/EcoR1 digest of a resilin clone;

FIG. 10 is a gel showing expression and purification of soluble Drosophilia pro-resilin in E. coli;

FIG. 11 is a gel illustrating the cross-linking of soluble pro-resilin with peroxidase enzymes has taken place;

FIG. 12 is a photograph of a sample of uncrossed-linked pro-resilin in test A and cross-linked resilin in test tube B;

FIG. 13 shows graphically the fluorescence spectrum of cross-linked resilin;

FIG. 14 gives the amino acid sequence for cloned recombinant resilin in accordance with the present invention; and

FIG. 15 shows the sedimentation equilibrium analysis of resilin which gives a molecular weight estimate of soluble resilin;

FIG. 16 is a gel demonstrating resilin production in the method of Example 4;

FIG. 17 is a gel showing pro-resilin production under different induction conditions;

FIG. 18 is a gel showing the fractions emerging from a nickel column and demonstrating purification of recombinant pro-resilin;

FIG. 19 is a gel demonstrating resilin production in an auto-induction method;

FIG. 20 is a gel demonstrating resilin production under a variety of growth conditions in the auto-induction method;

FIG. 21 is a gel showing that cross-linking takes place after one (1) hour of irradiation of a resilin solution with gamma radiation;

FIG. 22 is a gel showing that cross-linking of a resilin solution takes place after exposure to UVB radiation;

FIG. 23 is a gel showing cross-linking of resilin with UV radiation in the presence of riboflavin;

FIG. 24 is a gel showing fluorescein cross-linking of resilin with white light;

FIG. 25 shows the results of a further experiment with fluorescein cross-linking;

FIG. 26 shows the results of coumarin cross-linking with an ultraviolet mercury lamp as described in Example 14;

FIG. 27 plots percentage dityrosine cross-link formation from tyrosine residues in resilin against exposure time (in minutes) to white light when fluorescein is added to the resilin;

FIG. 28 is a gel showing photo-induced cross-linking of resilin exon 1 recombinant protein as described in Example 16. Irradiation was for ten (10) seconds. Lane 1: molecular weight standard; Lane 2: resilin only; Lane 3: resilin plus S₂0₈; Lane 4: resilin plus ((Ru)II)(pby₃)²⁺; Lane 5: resilin plus S₂0₈; plus ((Ru) II)(PBY₃)²⁺; and

FIG. 29 shows the effect of ((Ru(II)(bpy)³)²⁺ dilution on degree of soluble resilin (1 mg/ml in PBS) crosslinking. Lane 1: resilin+S₂O₈+((Ru(II)(bpy)³)²⁺ (no light); lane 2: resilin+S₂O₈; lane 4: resilin+((Ru(II)(bpy)³)²⁺; lane 5: resilin+S₂O₈+200 μM ((Ru(II)(bpy)³)²⁺; Lane 6: resilin+S₂O₈+100 μM ((Ru(II)(bpy)³)²⁺; lane 7: resilin+S₂O₈+50 μM ((Ru(II)(bpy)³)²⁺; Lane 8: resilin+S₂O₈+25 μM ((Ru(II)(bpy)³)²⁺; Lane 9: resilin+S₂O₈+12.5 μM ((Ru(II)(bpy)³)²⁺; Lane 10: resilin+S₂O₈+6.25 μM ((Ru(II)(bpy)³)²⁺; Lane 11: resilin+S₂O₈+3.125 μM ((Ru(II)(bpy)³)²⁺; Lane 12: resilin+S₂O₈+1.56 μM ((Ru(II)(bpy)³)²⁺;

FIG. 30 illustrates the homologies between resilin sequences from different insects;

FIG. 31 is a mass spectrum of a peptide in accordance with the invention; and

FIG. 32 is a graph comparing dityrosine fluorescence produced by various peroxidases.

### DETAILED DESCRIPTION OF THE INVENTION

The resilin gene (CG15920) was tentatively identified from the genome sequence of Drosophila melanogaster (Ardell, D H and Andersen, S O (2001), through analysis of the Drosophila genome database. The protein comprises short repeat sequences characteristic of other elastic proteins such as elastin and spider flagelliform silk, which are dominated by the VPGVG and GPGGX units, respectively. For these sequences it was suggested that they form beta-turns, and that the resulting series of beta turns forms a beta spiral (Ardell and Andersen, 2001), which can act as a readily deformed spring (a “nanospring”).

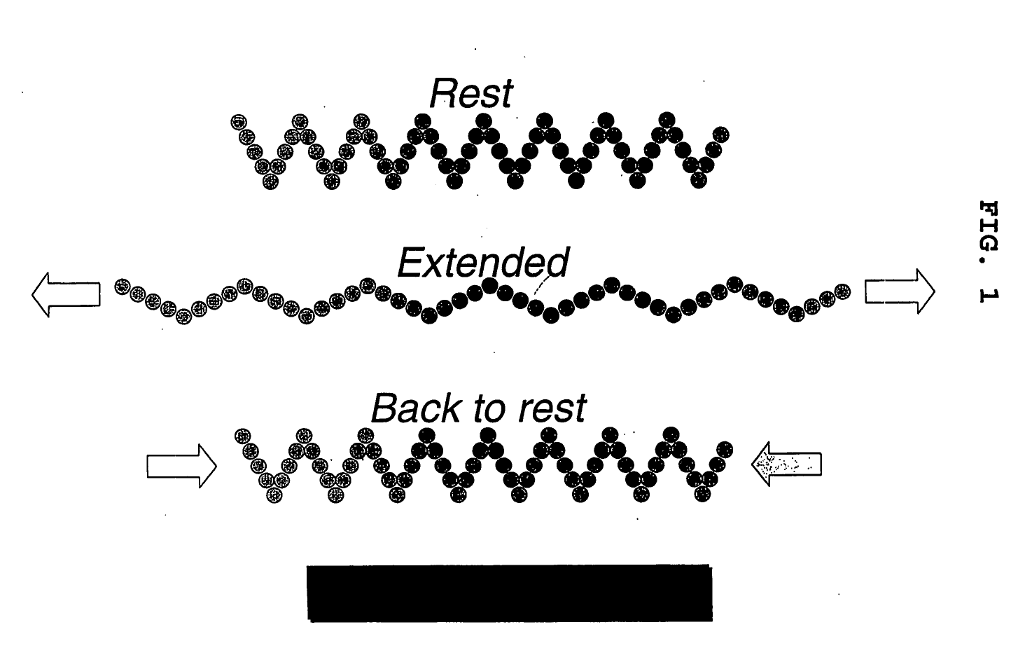

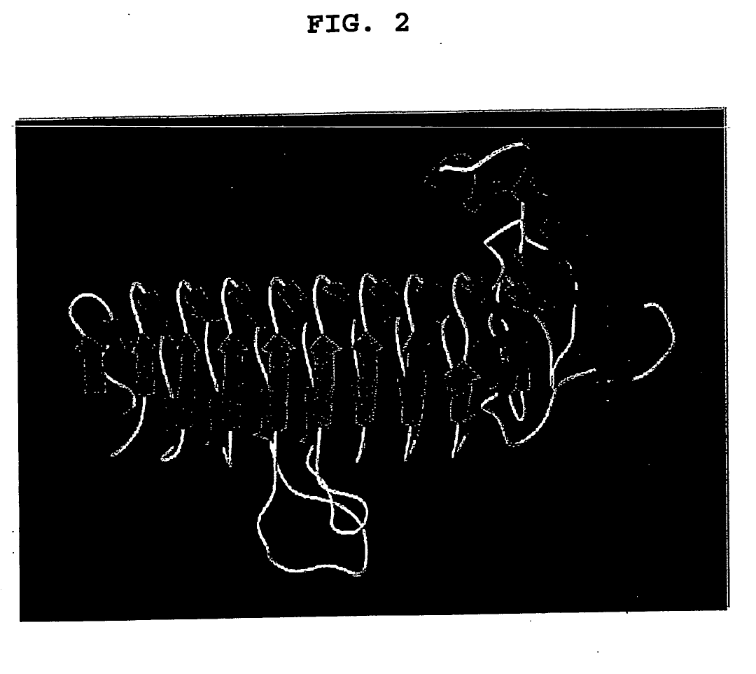

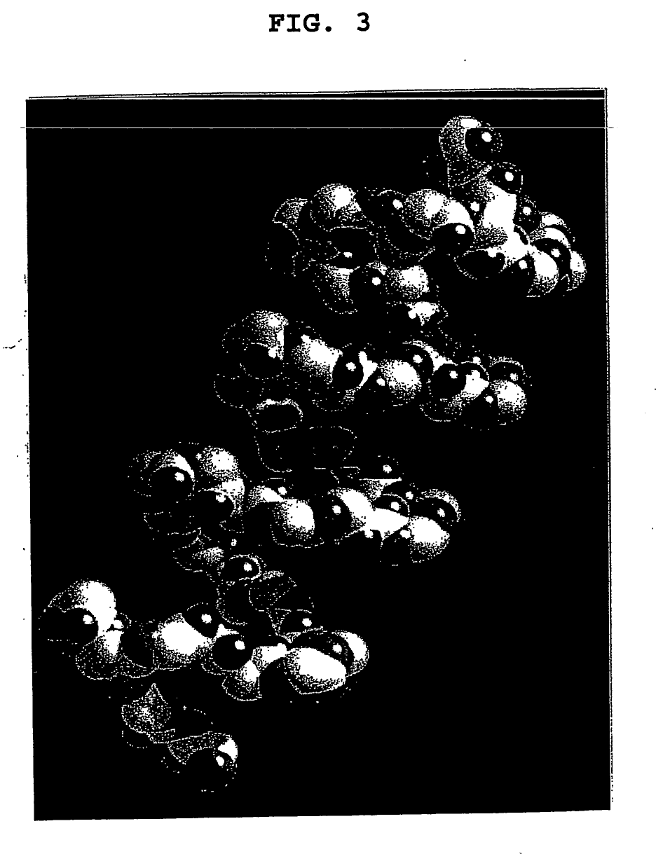

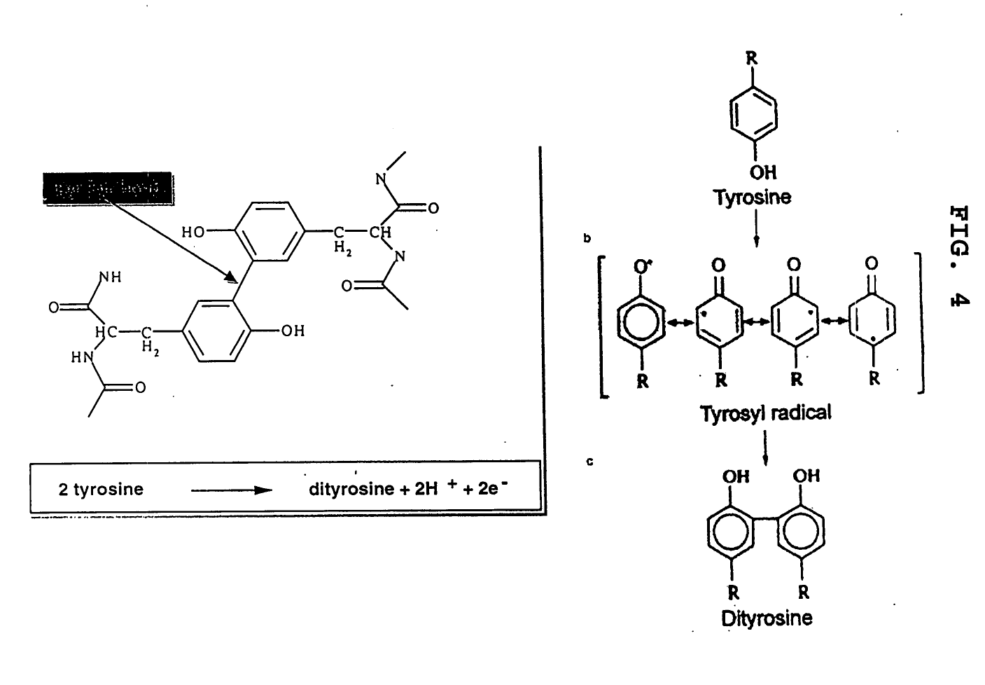

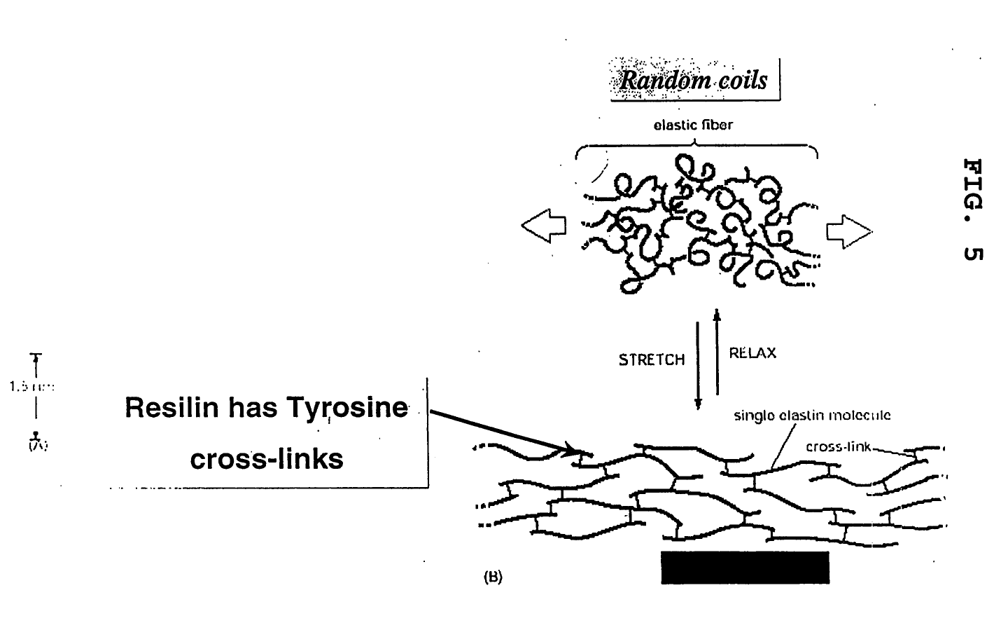

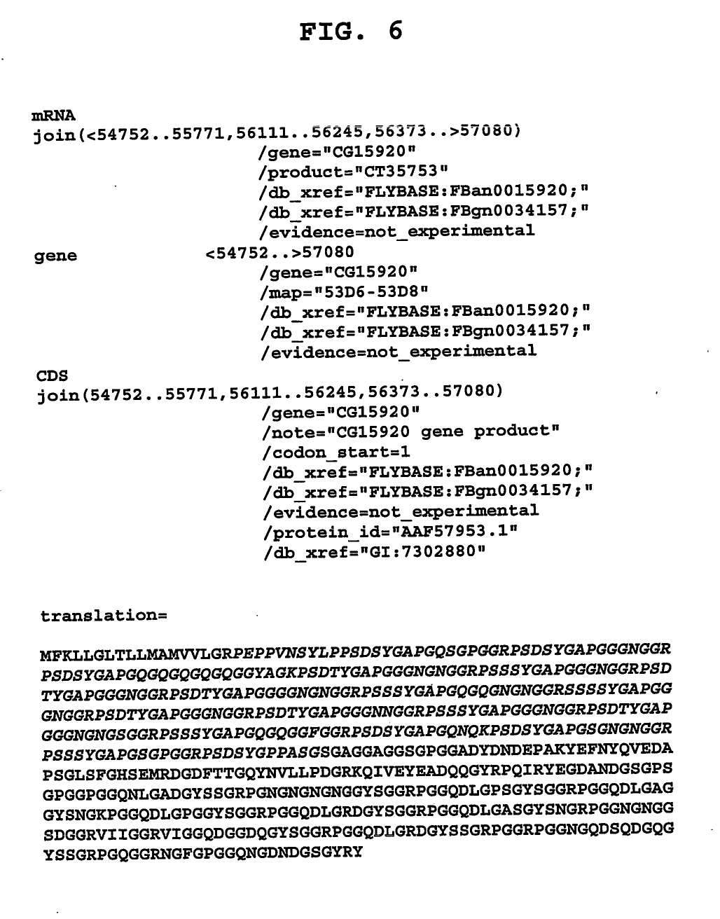

FIG. 1 shows schematically how a beta-spiral structure as in the present invention may revert from an extended position back to a rest position. This is an entropy-driven process to which the rubbery properties of elastomeric polypeptides is frequently attributed. FIGS. 2 and 3 show a typical beta-spiral structure (in this case from UDP-N-acetylglucosamine acyltransferase) which may extend and revert to a rest position in the manner illustrated in FIG. 1. The beta strands in FIG. 2 are represented by arrow structures. These are connected by a beta-turn motif, and these are generally initiated by a 2 amino acid sequence of PG or GG. The provision of a plurality of beta-turn motifs allows the beta-strands to form a beta-spiral of the type shown in FIG. 2 and, with a space filling model of a peptide from the HMW protein, in FIG. 3 (from: Parchment et al. (2001). Tyrosine is able to form dityrosine through a free radical mechanism, as illustrated in FIG. 4. The present inventors have been able to prepare a bioelastomer from resilin through formation of dityrosine cross-links between monomer units. Uncrossed-linked monomeric units are also useful in certain applications such as in nanomachines.

In a particularly preferred embodiment of the present invention the polypeptides are cross-linked to form an insoluble gel from a solution, preferably one with a relatively high concentration of protein, more preferably a protein concentration greater than 10% w/v. The person skilled in the art will appreciate that solutions with a higher concentration of protein may be effectively cross-linked but economic considerations dictate that very high concentrations of protein will not be used, and that there is a limit to the concentration of protein which will remain in solution. Likewise, solutions with a lesser concentration of protein may be cross-linked although the gel resulting from this procedure may be less effective.

Any means of cross-linking may be employed provided that the dityrosine bonds are formed. These methods are well known to the person skilled in the art and are discussed by Malencik and Anderson (1996), the contents of which are incorporated herein by reference.

In an embodiment enzyme-mediated cross-linking may be employed. Although peroxidases such as horseradish peroxidase and lactoperoxidase can form dityrosine cross-links between proteins, their specific activity towards tyrosine residues is only about 1% of the activity displayed by the Arthromyces peroxidase. This unique property of the fungal enzyme was identified and used by Malencik and Anderson (1996) to cross-link calmodulin (which contains only two Tyr residues) into a very large MW polymer.

Other systems can also be used to cross-link protein molecules via di-tyrosine cross-links. These include:

Other peroxidases could also be used to cross-link the soluble resilin into a polymer. These include:
A. Duox peroxidase from Caenorhabditis elegans which is responsible for the cross-linking of tyrosine residues in the cuticle. This enzyme has been shown to cause formation of dityrosine in worm cuticle proteins (Edens et al. 2001).
B. Sea urchin ovoperoxidases play an important role in hardening the egg membranes immediately following fertilisation. The genes encoding these enzymes have been cloned from two species of sea urchins (LaFleur, et al. 1998). Chorion peroxidase from mature eggs of the mosquito Aedes aegypti eggs. (Nelson et al. 1994). This chorion peroxidase has a specific activity 100 times greater than horseradish peroxidase to tyrosine. The enzyme was shown to catalyse polypeptide and chorion protein cross-linking through dityrosine formation in vitro. The enzyme is responsible for chorion formation and hardening. In a further embodiment the PICUP (photo-induced-cross-linking of unmodified proteins) reaction, which is induced by very rapid, visible light photolysis of a tris-bipyridyl Ru(I) complex in the presence of an electroniceptor may be used to induce cross linking (Fancy and Kodadek, 1999).

Following irradiation, a Ru(III) ion is formed, which serves as an electron abstraction agent to produce a carbon radical within the polypeptide, preferentially at a tyrosine residue, and thus allows dityrosine link formation. This method of induction allows quantitative conversion of soluble resilin or pro-resilin fragments to a very high molecular weight aggregate. Moreover this method allows for convenient shaping of the bioelastomer by introducing recombinant resilin into a glass tube of the desired shape and irradiating the recombinant resilin contained therein.

In a further embodiment, gamma-irradiation may be employed for cross-linking resilin monomers, although care must be taken not to damage the protein through exposure to this radiation. UVB radiation cross-linking may also be undertaken in the presence of absence of riboflavin. In the absence of riboflavin a substantial amount of cross-linking takes place within one hour of exposure, but this time is substantially reduced if riboflavin is present. Still further, cross-linking may be effected with ultra-violet light in the presence of coumarin or by white light in the presence of fluorescein. An analysis of the dityrosine may be performed using conventional methods such as high performance liquid chromatography measurements in order to ascertain the extent of dityrosine cross-link formation.

To determine the effect of cross-links and the optimal number of cross-links per monomer unit, the resilience of a cross-linked polymer can be measured using methods known in the art. The level of cross-linking can vary provided that the resulting resilin repeat polymer displays the requisite resilient properties. For example, when the cross-linking is by gamma-irradiation, the degree of cross-linking is a function of the time and energy of the irradiation. The time required to achieve a desired level of cross-linking may readily be computed by exposing non-cross-linked polymer to the source of radiation for different time intervals and determining the degree of resilience (elastic modulus) of the resulting cross-linked material for each time interval. By this experimentation, it will be possible to determine the irradiation time required to produce a level of resiliency appropriate for a particular application (see, e.g., U.S. Pat. No. 4,474,851, the contents of which are incorporated herein by reference).

The resilin repeat polymers are preferably lightly cross-linked. Preferably, the extent of cross-linking is at least about one cross-link for every five or ten to one hundred monomer units, e.g., one cross-link for every twenty to fifty monomer units. Indeed, we have found that about 18% of the available tyrosine in the proresilin monomer is converted to dityrosine following enzymatic oxidation of proresilin.

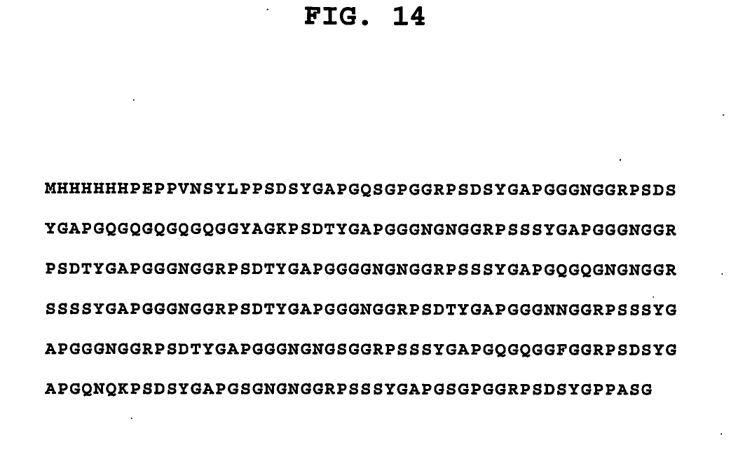

The extent of cross-linking may be monitored during the reaction or pre-determined by using a measured amount of reactants. For example, since the dityrosine cross-link is fluorescent, the fluorescence spectrum of the reactant mixture may be monitored during the course of a reaction to determine the extent of cross-linking at any particular time. This is illustrated in FIG. 14, and allows for control of the reaction and the properties of the bioelastomer which results. Once the desired level of cross-linking is achieved (indicated by reaching a predetermined fluorescence intensity) a peroxidase-catalysed reaction may be quenched in a manner known to the person skilled in the art.

For example, glutathione can be added or the gel can be soaked in a solution of glutathione and glutathione peroxidase as described in Malencik and Anderson (1996).

Fusion proteins may be produced through cloning techniques known to the person skilled in the art. Alternatively, other means of linking molecules may be employed including covalent bonds, ionic bonds and hydrogen bonds or electrostatic interactions such as ion-dipole and dipole-dipole interactions. The linkage may be formed, for example, by the methods described above for cross-linking of the resilient component. It may be necessary to provide appropriate chemical moieties in the second component to allow cross-linking with the first, resilient component. Such moieties are well known to the person skilled in the art and include, for example, amino, and carboxylic groups. Where the second component is a protein, the association between the components can be effected by recombinant nucleic acid technology.

A hybrid resilin molecule can contain various numbers of both components. For example they can contain (a) one molecule of each component, (b) one molecule of the first component and a plurality of molecules (e.g., two to five hundred or ten to one hundred) of the second component, (c) a plurality of molecules of the first component and one molecule of the second component, or (d) a plurality of molecules of both components. Optimal numbers and positioning of inserted sequences can be determined by the person skilled in the art. The degree of linkage between the two components and the relative number of each component in the final hybrid resilin molecule can be varied so as to provide the desired level of the function of both components. The hybrid resilin molecules include those in which the fragments of the second component are inserted within the sequence of the resilin polypeptide. Alternatively, resilin repeat sequences can be inserted in the second component molecules. The inserted sequences can be inserted tandemly or alternately.

For example, to make biomaterials that require strength as well as resilience, a first component can be combined with a load-bearing second component. Examples of naturally occurring load-bearing polymers are collagen and silk or silk-like proteins, e.g., insect (or spider)-derived silk proteins. Other suitable types of polymers that could used as second components to endow strength include polyamides, polyesters, polyvinyls, polyethylenes, polyurethanes, polyethers, and polyimides. Hybrid resilin molecules that include such polymers have a variety of uses including, for example, artificial joint ligaments with increased resilience where the second component is collagen or a functional fragment thereof. Functional fragments of collagen include those with the following sequence: Gly-Pro-Hyp, where Hyp is hydroxyproline.

Alternatively, by using silk worm, an insect or spider silk protein (e.g., fibroin) or a functional fragment thereof, as the second component, an extremely light-weight, resilient, and durable thread or filament can be produced, which can be woven into a fabric. Such fabrics are useful in the manufacture, for example, of military clothing. Fragments of fibroin include those with the following sequences: Gly-Ala-Gly-Ala-Gly-Ser, Ala-Ser-Ala-Ala-Ala-Ala-Ala-Ala, Ser-Ser-Ala-Ala-Ala-Ala-Ala-Ala-Ala-Ala, and Ala-Ala-Ala-Ala-Ala-Ala-Ala-Ala.

The materials of the invention, i.e., resilin repeat polymers, or hybrid resilin molecules, can be manufactured in various useful physical forms, e.g., woven or non-woven sheets, gels, foams, powders, or solutions. Furthermore, where desired, the materials, during manufacture, can be molded into appropriate shapes as, for example, in the case of medical prostheses such as vascular prostheses or joint prostheses.

When used in vivo, and in particular inside the body of a subject, e.g., a human patient, it is important that the material be biocompatible. A “biocompatible” material is not substantially mutagenic, antigenic, inflammatory, pyrogenic, or hemolytic. Furthermore, it must neither exhibit substantial cytotoxicity, acute systemic toxicity, or intracutaneous toxicity, nor significantly decrease clotting time. In vivo and in vitro tests for these undesirable biological activities are well known in the art; examples of such assays are given, for example, in U.S. Pat. No. 5,527,610, the contents of which are incorporated by reference. Also, when used in vivo, the materials may be biogradable.

In light of their high glycine content, insolubility, chemical inertness and biodegradability, the resilin polypeptides and hybrid molecules used for in vivo applications (e.g., prostheses and tissue adhesion-preventing barriers) are likely to be substantially biocompatible. In the event that toxicity or immunogenicity, for example, occurs in a relevant material, methods for modulating these undesirable effects are known in the art. For example, “tanning” of the material by treating it with chemicals such as aldehydes (e.g., glutaraldehyde) or metaperiodate will substantially decrease both toxicity and immunogenicity. Preferably, the materials used to make devices for in vivo use are also sterilizable.

Resilin may be used to produce nanomachines and biosensors.

The entropy-driven extension and resilience of resilin, can be used in a number of nanomachine applications, including:
(A) MEMS applications of nanomachines. Significant improvements in micro-electro-mechanical device functions. Response times of such devices can be as short as milliseconds.
(B) Biosensor applications such as sensing the binding of drugs, xenobiotics and toxic chemical compounds. The nanomachine envisaged comprises an elastomer, such as resilin, coupled in series to a hydrophobically folded globular receptor protein. For example, it has been shown (Urry, 2001) that binding of one phosphate residue (to a kinase recognition sequence such as RGYSLG) per 300 residues of a repeat sequence in the elastomer titin, causes complete hydrophobic unfolding of the titin β-barrel. This would cause an increase in the contour length which could be measured.
(C) Acoustic absorption properties of the β-barrel nanomachine elastomer sas described by Urry (2001).

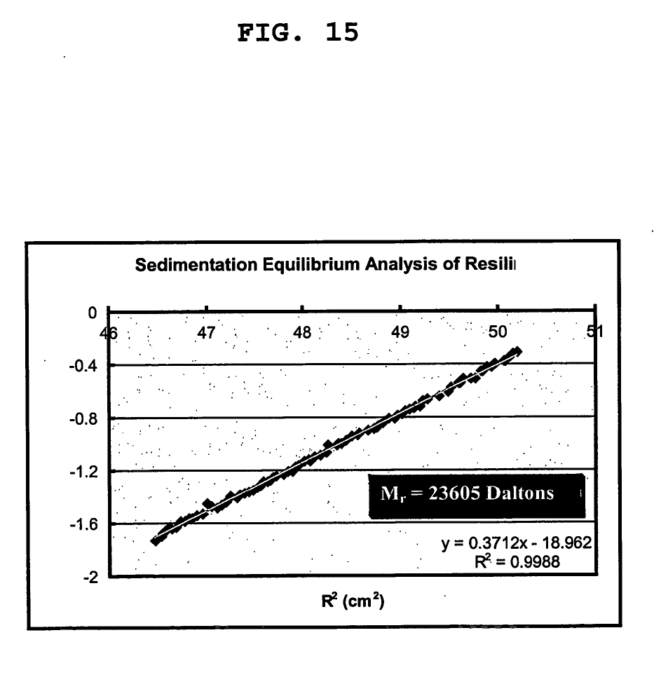

Polypeptides of the present invention such as that derived from the first exon of the resilin gene, whose sequence is given in FIG. 15, can be prepared in any suitable manner. While chemical synthesis of such polypeptides is envisaged, it is preferred to transform an appropriate host cell with an expression vector which expresses the polypeptide. The design of a host-expression vector system is entirely within the capability of the person skilled in the art.

The expression systems that can be used for purposes of the invention include, but are not limited to, microorganisms such as bacteria (for example, E. coli including but not limited to E. coli strains BL21 (DE3) plysS, BL21 (DE3)RP and BL21* and B. subtilis) transformed with recombinant bacteriophage DNA, plasmid DNA, or cosmid DNA expression vectors containing the nucleotide sequences; yeast transformed with recombinant yeast expression vectors; insect cells infected with recombinant viral expression vectors (baculovirus); plant cell systems infected with recombinant viral expression vectors (e.g., cauliflower mosaic virus, CaMV; tobacco mosaic virus, TMV) or transformed with recombinant plasmid expression vectors; or mammalian cells (e.g., COS, CHO, BHK, 293, 3T3) harboring recombinant expression constructs containing promoters derived from the genome of mammalian cells (e.g. metallothionein promoter) or from mammalian viruses.

In bacterial systems, a number of expression vectors may be advantageously selected depending upon the use intended for the gene product being expressed. For example, when a large quantity of such a protein is to be produced vectors which direct the expression of high levels of fusion protein products that are readily purified may be desirable. Such vectors include, but are not limited to, the E. coli expression vector pETMCS1 (Miles et al, 1997), pUR278 (Ruther et al., EMBO J., 2:1791, 1983), in which the coding sequence may be ligated individually into the vector in frame with the lacZ coding region so that a fusion protein is produced; pIN vectors (Inouye & Inouye, Nucleic Acids Res., 13:3101, 1985; Van Heeke & Schuster, J. Biol. Chem., 264:5503, 1989); and the like. pGEX vectors may also be used to express foreign polypeptides as fusion proteins with glutathione S-transferase (GST). In general, such fusion proteins are soluble and can easily be purified from lysed cells by adsorption to glutathione-agarose beads followed by elution in the presence of free glutathione. The pGEX vectors are designed to include thrombin or factor Xa protease cleavage sites so that the cloned target gene product can be released from the GST moiety.

In mammalian host cells, a number of viral-based expression systems can be utilized. In cases where an adenovirus is used as an expression vector, the nucleotide sequence of interest may be ligated to an adenovirus transcription/translation control complex, e.g., the late promoter and tripartite leader sequence. This chimeric gene can then be inserted in the adenovirus genome by in vitro or in vivo recombination. Insertion in a non-essential region of the viral genome (e.g., region E1 or E3) will result in a recombinant virus that is viable and capable of expressing the gene product in infected hosts (e.g., See Logan & Shenk, Proc. Natl. Acad. Sci. USA, 81:3655, 1984). Specific initiation signals may also be required for efficient translation of inserted nucleotide sequences. These signals include the ATG initiation codon and adjacent sequences. In cases where an entire gene or cDNA, including its own initiation codon and adjacent sequences, is inserted into the appropriate expression vector, no additional translational control signals may be needed. However, in cases where only a portion of the coding sequence is inserted, exogenous translational control signals, including, perhaps, the ATG initiation codon, must be provided. Furthermore, the initiation codon must be in phase with the reading frame of the desired coding sequence to ensure translation of the entire insert. These exogenous translational control signals and initiation codons can be of a variety of origins, both natural and synthetic. The efficiency of expression can be enhanced by the inclusion of appropriate transcription enhancer elements, transcription terminators, etc. (Bittner et al., Methods in Enzymol., 153:516, 1987).

In addition, a host cell strain can be chosen which modulates the expression of the inserted sequences, or modifies and processes the gene product in the specific fashion desired. Such modifications (e.g., glycosylation and generation of Hyp and DOPA residues) and processing (e.g., cleavage) of protein products can be important for the function of the protein. Appropriate cell lines or host systems can be chosen to ensure the correct modification and processing of the foreign protein expressed. Mammalian host cells include but are not limited to CHO, VERO, BHK, HeLa, COS, MDCK, 293, 3T3, and WI38.

For long-term, high-yield production of recombinant proteins, stable expression is preferred. For example, cell lines which stably express the sequences described above can be engineered. Rather than using expression vectors which contain viral origins of replication, host cells can be transformed with DNA controlled by appropriate expression control elements (e.g., promoter, enhancer sequences, transcription terminators, polyadenylation sites, etc.), and a selectable marker. Following the introduction of the foreign DNA, engineered cells can be allowed to grow for 1-2 days in an enriched medium, and then are switched to a selective medium. The selectable marker in the recombinant plasmid confers resistance to the selection and allows cells to stably integrate the plasmid into their chromosomes and grow to form foci which in turn can be cloned and expanded into cell lines. This method can advantageously be used to engineer cell lines which express the gene product. Such engineered cell lines can be particularly useful in screening and evaluation of compounds that affect the endogenous activity of the gene product.

A fusion protein can be readily purified by utilizing an antibody or a ligand that specifically binds to the fusion protein being expressed. For example, a system described by Janknecht et al., Proc. Natl. Acad. Sci. USA, 88:8972, 1991) allows for the ready purification of non-denatured fusion proteins expressed in human cell lines. In this system, the gene of interest is subcloned into a vaccinia recombination plasmid such that the gene's open reading frame is translationally fused to an amino-terminal tag consisting of six histidine residues. Extracts from cells infected with recombinant vaccinia virus are loaded onto Ni²⁺ nitriloacetic acid-agarose columns and histidine-tagged proteins are selectively eluted with imidazole-containing buffers. If desired, the histidine tag can be selectively cleaved with an appropriate enzyme.

In addition, large quantities of recombinant polypeptides can advantageously be obtained using genetically modified organisms (e.g., plants or mammals), wherein the organisms harbor exogenously derived transgenes encoding the polypeptide of interest (Wright et al., Bio/technology, 5:830, 1991; Ebert et al., Bio/technology, 9:835, 1991; Velander et al., Proc. Natl. Acad. Sci. USA, 89:12003, 1993; Paleyanda et al., Nature Biotechnology, 15:971, 1997; Hennighausen, Nature Biotechnology, 15:945, 1997; Gibbs, Scientific American, 277:44, 1997). The polypeptide of interest is expressed in a bodily tissue and then is purified from relevant tissues or body fluids of the appropriate organism. For example, by directing expression of the transgene to the mammary gland, the protein is secreted in large amounts into the milk of the mammal from which it can be conveniently purified (e.g., Wright et al., cited supra, Paleyanda et al., cited supra; Hennighausen, cited supra).

### EXAMPLES

### Example 1

### Cloning of the Resilin Gene from Drosophila melanogaster

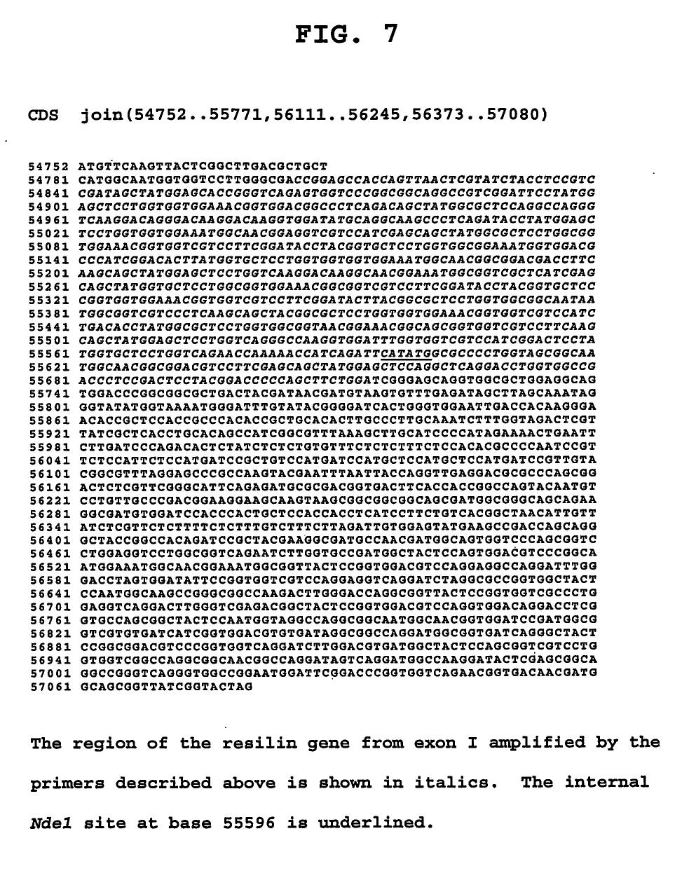

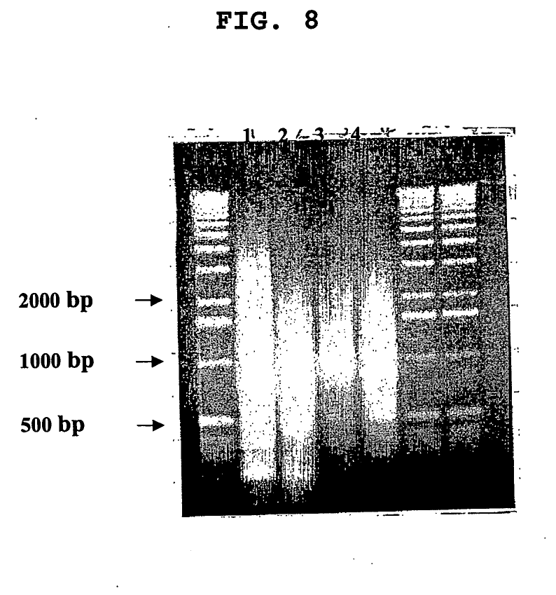

The first exon of the resilin gene (FIG. 6) was amplified from Drosophila melanogaster genomic DNA via PCR using two primers designed from the known DNA sequence (His)₆ coding sequence and an NdeI site while the reverse primer contained an EcoRI site. These restriction sites were included to facilitate cloning of the PCR product into the NdeI/EcoRI site of the E. coli expression vector pETMCS1 (Miles et al. 1997). The PCR product shown in FIG. 8, lane 3, was purified from the agarose gel using a commercial kit (MN) and cloned into the cloning vector pCR-Blunt (Invitrogen). The sequence of the insert was determined using dye-terminator nucleotide mixes (Big Dye-ABI). The sequence was found to be identical to that reported for the CG15920 sequence from Drosophila. An internal NdeI site was found at base 55596 (underlined in FIG. 7).

PCR primers were (forward) ResF3 and (reverse) RespepR1. The sequences of the primers were:

ResF3:
5′ . . . CCCATATGCACCATCACCATCACCATCCGGAGCCACCAGTT
AACTCGTATCTACC . . . 3′
RespepR1:
5′ . . . CCGAATTCCTATCCAGAAGCTGGGGGTCCGTAGGAGTCGGA
GGG . . . 3′

### Example 2

### Expression and Purification of the First Exon of the Resilin Gene from Drosophila melanogaster

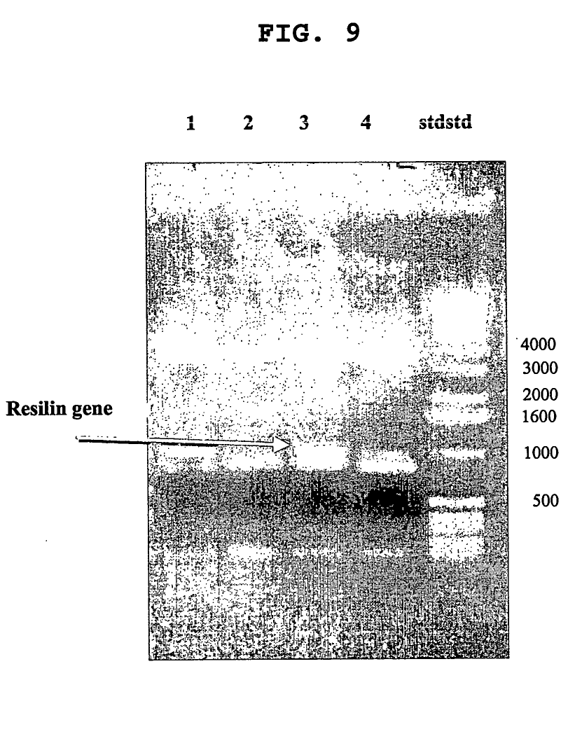

The sequence obtained above was obtained by partial digestion of the resilin/pCRBlunt clone with EcoRI/NdeI. The upper band (see FIG. 9) was excised from the gel and purified using a commercially available kit (Machery-Nagel) and ligated into the EcoI/NdeI site of the expression vector pETMCS1, using standard ligation conditions with T4 DNA ligase. About 200 ng of insert was ligated to 50 ng of vector at 12° C. overnight. The ligated recombinant plasmid mix was used to transform competent cells of the E. coli strain Top10 (Invitrogen) with selection for resistance to ampicillin (100 μg/ml) on Luria Broth (LB) agar plates. Colonies were selected and commercial kit (Machery-Nagel). The sequence of the expected recombinant plasmid insert was confirmed by DNA sequence analysis and matched the published sequence of CG15920.

The correct recombinant plasmid containing the Drosophila melanogaster resilin exon I sequence cloned into the NdeI/EcoRI site of expression vector pETMCS1 was isolated from a 2 ml overnight culture of the E. coli Top10 strain carrying this plasmid. This purified plasmid was then used to transform the E. coli strains BL21(DE3)plysS or the E. coli rne (BL21*) strain, with selection for resistance to both ampicillin (100 μg/ml) and chloramphenicol (34 μg/ml).

Small scale inductions of the recombinant protein were carried out by growing the two strains overnight in LB medium and the level of resilin recombinant protein production was compared to the E. coli and vector proteins expressed in E. coli BL21(DE3)plysS transformed with the vector pETMCS1 only. The results showed that the E. coli ribonuclease E mutant strain, BL21 Star™, (DE3)pLysS: F-ompT hsdS B (rB-mB-) gal dcm rne131 (DE3) pLysS (Cam R) contained more soluble recombinant resilin than the BL21(DE3)plysS strain (data not shown).

This recombinant BL21 Stare strain (resilin5/BL21Star) was therefore chosen for large-scale expression of the resilin recombinant protein.

### Example 3

### Scale-Up of Resilin Production

3 litres of LB medium (1 litre of medium in each of 3×2-litre baffled Ehrlenmeyer flask) was inoculated with an overnight culture of (resilin5/BL21Star) to an A600 of 0.1. The cells were grown with vigorous aeration (200 cycles per minute) on a rotary shaker at 37° C. until the A600 reached 0.8. At this point, IPTG (isopropyl-β-D-thiogalactopyranoside) was added to 1 mM final concentration and the culture was grown for a further 4.5 h at 37° C. with vigorous aeration. The cells were harvested by centrifugation (10,000×g 20 min at 4° C.). The cell pellets were resuspended at 4° C. in 80 ml of 50 mM NaH₂PO₄/Na₂HPO₄ buffer containing 150 mM NaCl and 1× protease inhibitor cocktail (EDTA-free) (Roche—Cat. No. 1 873 580). The cells were disrupted with a sonicator (4×15 sec bursts) following addition of Triton X-100 (to 0.5% final conc).

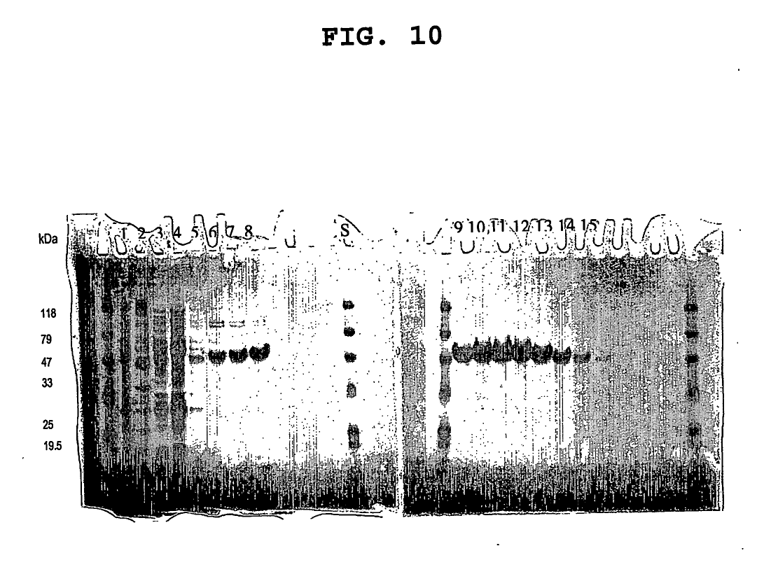

Membrane and soluble fractions were separated by centrifugation of the disrupted cells at 100,000×g for 1 h at 4° C. The soluble fraction was bound to a 10 ml packed column of Ni-NTA affinity resin (Qiagen—Ni-NTA Superflow (25 ml) 25 ml nickel-charged resin (max. pressure: 140 psi) (cat #30410) for 1.5 h at 4° C. The resin was packed into a column which was washed (at 1 ml/min) with loading buffer (50 mM NaH₂PO₄/Na₂HPO₄ buffer containing 150 mM NaCl) until the A₂₈₀ fell to near baseline and stabilised. In order to remove E. coli proteins bound non-specifically to the resin, buffer containing 10 mM imidazole was passed through the column, resulting in elution of many E. coli proteins. Once the A₂₈₀ had fallen to near baseline, a 10 mM-150 mM gradient of imidazole in loading buffer was passed through the column at 2.0 ml/min. Fractions (2 ml) were collected and 10 μl aliquots of each fraction were analysed by SDS-PAGE. The gel was stained with Coomassie blue and destained (10% acetic acid, 30% ethanol) to reveal the affinity column chromatographic purification of soluble recombinant resilin protein. The fractions containing purified resilin (fractions 12-48) were pooled and concentrated to about 20 ml volume and dialysed against a buffer containing 50 mM Tris/HCl 100 mM NaCl pH 8.0. The dialysed protein solution was then concentrated using a Centricon™ filtration device (MW cutoff=10,000 Da) to a protein concentration of 80 mg/ml, 150 mg.ml or 250 mg/ml (by A₂₈₀ measurement). The results of this affinity column purification of soluble resilin is shown in FIG. 10.

The molecular weight of the soluble recombinant resilin was shown by SDS-PAGE to be ca. 46,000 Da, which suggested that the recombinant protein might be produced in E. coli as a dimer, since the calculated MW of the 303 amino acid protein is 28,466 Da. However, when a sample (A280=0.4) dialysed against TBS, was analysed by equilibrium density gradient ultracentrifugation, the results clearly showed that the calculated thermodynamic molecular weight of the soluble recombinant protein was 23,605 Da (FIG. 15). We can conclude that the recombinant resilin expressed from the first exon of the CG15902 gene from Drosophila melanogaster is a monomer.

### Example 4

### Growth of E. coli on LB Medium: Recombinant Resilin Production

6 litres of LB broth is prepared with distilled water using 2×LB EZMix (Sigma). The pH is adjusted to 7.5 with 1M NaOH. Trace elements are added at 0.25 ml per litre of broth and phosphate buffer at 10 ml per litre of broth. The broth is added to 6×2 L baffled flasks and autoclaved.

Trace Elements Mix:

| | |
|---|---|
| FeCl₃•6H₂O | 2.713 g |
| CuCl₂ | 0.101 g |
| CoCl₂•6H2O | 0.204 g |
| H₃BO₄ | 0.051 g |
| Na₂MoO₄•2H₂O | 0.202 g |
| CaCl2•2H2O | 0.0977 g |
| ZnSO4•7H2O | 0.300 g |

Conc HCl 10 ml—make up to 100 ml with H₂O. Use 25 μL per 100 ml culture.

A single colony of the recombinant E. coli strain is added to 400 ml broth with 0.4 ml Ampicillin (100 mg/ml) and 0.4 ml Chloramphenicol (34 mg/ml) in a laminar flow cabinet to ensure sterile conditions. The broth is shaken at 220 rpm overnight at 37° C.

The following morning, the OD₆₀₀ of the overnight culture is measured. An aliquot of the overnight culture is added to the 6 litres of broth to give a final OD₆₀₀ of 0.15. 1 ml of both Ampicillin and Chloramphenicol (same concentrations as above) is added to each 1 litre of broth along with 1 drop (ca. 50 μl) of Antifoam 289 (Sigma). The broth is shaken at 220 rpm for 2 hours until the OD₆₀₀ is around 1.0. At this time, 0.5 ml of 1M IPTG is added to each litre of broth followed by shaking for another 3 hours.

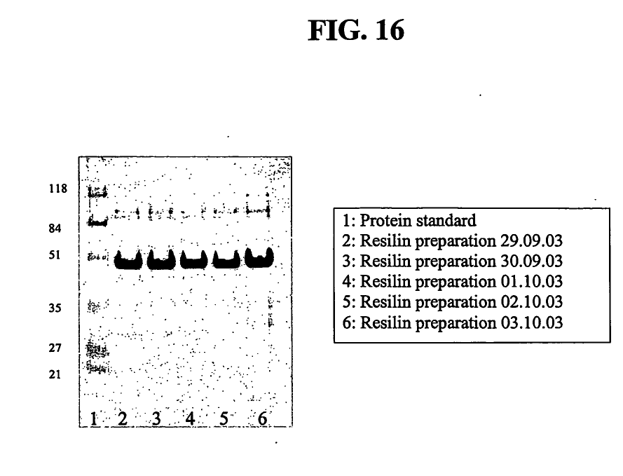

The cells from the culture are collected by centrifugation at 6000 rpm at 4° C. for 20 minutes. The supernatant is discarded and the pellet removed and kept in the −80° C. freezer, ready for processing. A 40 ml sample is spun and the small pellet kept in the −80° C. freezer until ready for processing. The small pellet is used to verify the resilin content of the cells. This pellet from a 40 ml culture is processed through cell lysis and affinity chromatography on a Ni-NTA resin (Qiagen). A typical result is shown in FIG. 16.

Change of Inducing Conditions

The inducing conditions were changed by inducing for 4 hours and inducing after the culture had reached OD₆₀₀=2, to determine the effect on the resilin yield.

Three 40 ml broths were autoclaved as per usual recipe. Each was induced at different times and OD₆₀₀ values according to the conditions below:

1: Induced at OD₆₀₀=1 for 3 hours (usual conditions)

2: Induced at OD₆₀₀=1 for 4 hours

3: Induced at OD₆₀₀=2 for 3 hours

The cultures were induced with 20 μl of 1M IPTG.

All other conditions and recipes etc remained the same as per the usual recipe. The culture was spun and the pellet resuspended in 1 ml of phosphate buffer with 0.1% Triton-X100 (TX-100) and protease inhibitor, keeping the same final OD₆₀₀ ratios. After sonication and spinning, the resulting supernatant was put through a Nickel column. The resulting eluate was run on an SDS-PAGE gel. The results are shown below.

| | Condition 1 | Condition 2 | Condition 3 |
|---|---|---|---|
| Final OD₆₀₀ | 2.042 | 1.917 | 2.278 |
| Resuspension volume (ml) | 1.02 | 0.95 | 1.15 |

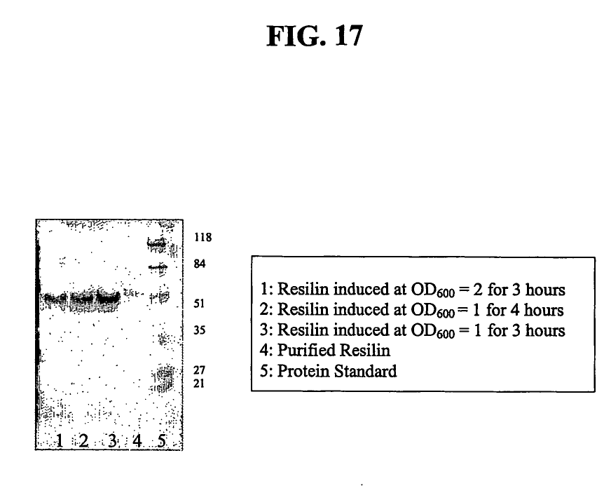

The results given in FIG. 17 show that there is little difference between the resilin yields for the various conditions. If anything, the usual conditions of inducing at OD₆₀₀=1 for 3 hours appears to be better than the other variations. Whilst the gel does not give quantitative results, it does show that there is no significant gains achieved by altering the inducing conditions.

### Example 5

### Alternative Strain of E. coli

The E. coli BL21 (DE3)plysS strain was compared to BL21 (DE3) RP strain of E. coli to determine if we could improve our production of resilin. This strain contains plasmid-encoded copies of tRNA genes which can overcome rare Arg and Pro codons.

The resilin expression clone (resilin 5) DNA was transformed into another strain of E. coli, the BL21 (DE3) RP strain (Stratagene). This strain is expected to give better production due to the ability to produce rare codons. To determine the resilin production characteristics, three 40 ml LB broths were autoclaved. One broth contained the Resilin 5 strain, one with Resilin RP strain and the other with the vector alone. The vector was included because it would not produce resilin and hence would help ensure that the results were valid.

The three cultures were grown with a starting OD₆₀₀=0.15, inducing with 0.5 mM IPTG at OD₆₀₀ approximately equal to 0.8. After 3 hours of shaking at 37° C., the final OD₆₀₀ of each culture was measured. After spinning, the resulting pellet was resuspended to ensure the same relative OD₆₀₀ in phosphate buffer+0.1% TX-100+protease inhibitor, as the final OD₆₀₀ reading. 100 μl of 25 mg/ml lysozyme was added to the Resilin RP before sonication.

| | Final OD₆₀₀ | Resuspension Volume |
|---|---|---|
| Resilin 5 | 1.326 | 10 ml |
| Resilin RP | 0.323 | 2.5 ml |
| Vector | 1.206 | 10 ml |

After spinning, the supernatant was treated with Nickel resin and the eluate run on an SDS-PAGE gel.

The resilin band is strong in Resilin 5 however it appears somewhat weaker in Resilin RP suggesting that the Resilin 5 strain more effectively produces the resilin protein. From this, we can conclude that there is no advantage to producing resilin in the RP strain compared with the BL21 Star strain.

### Example 6

### Alternative Procedure for Production of Recombinant Resilin in E. coli

The medium used for autoinduction of the recombinant resilin gene was the Overnight Express Autoinduction System™ (Novagen).

### Procedure 1

Add 1 ml of overnight culture (OD₆₀₀ approximately 6.0) to the culture medium and shake for 4 hours at 37° C. at 220 rpm. Shake for a further 26 hours at room temperature. Spin as per usual method.

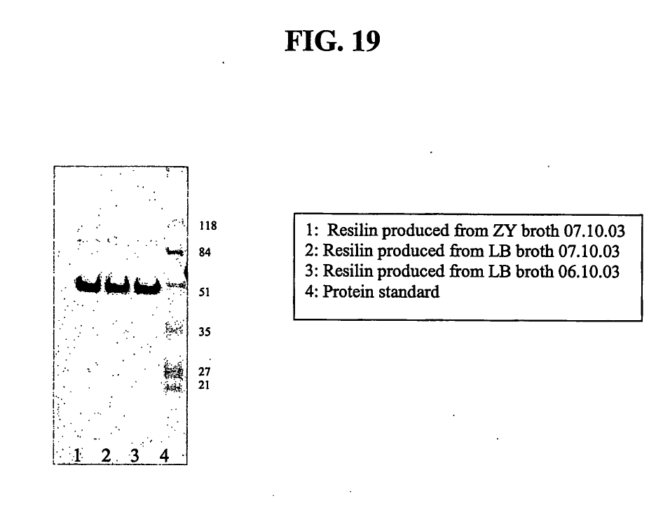

Using this method, the final OD₆₀₀ is approximately 12.0 rather than the usual 4.0 obtained on LB medium. A 40 ml sample was spun and processed to compare with resilin produced from LB broth. The results are shown in FIG. 19. Since the pellet from the 40 ml spin was 3.6 times greater in weight than the usual resilin pellet, it was resuspended in 3.6 ml rather than the 1 ml used for the usual 40 ml pellet. The resuspended pellets were sonicated and spun. 1 ml of the resulting supernatant was processed through a Nickel column and the elution was run on a gel. The gel shows that the production of resilin is equivalent to that from the LB broth method. Since we are achieving approximately 4 times the number of cells per litre of broth, we are effectively increasing our productivity 4-fold.

### Procedure 2

As per Procedure 1 however add overnight culture to broth at approximately 3.30 pm and shake overnight at 37° C. Cells were collected by centrifugation the following morning after 18 hours growth.

Variation of Growth Conditions

Several alternative recipes and procedures were tested on 50 mL broths to determine the optimal method for producing resilin:
A: standard test conditions, no variations
B: 100 mL of starting culture
C: 500 mL of starting culture
D: 100 mL of starting culture and double the concentration of growth medium.
E: Culture grown overnight (18 hrs) at 37° C.
G: Secondary temperature was 23° C.

H: Culture grown overnight (4.00 pm-2.00 pm the following afternoon) at 37° C.

| | OD₆₀₀ @ 8.10 am | OD₆₀₀ @ 9.50 am | OD₆₀₀ @ 2.00 pm |
|---|---|---|---|
| A | 6.794 | 7.146 | 7.188 |
| B | 6.618 | 7.038 | 7.546 |
| C | 7.036 | 7.872 | 8.162 |
| D | 5.768 | 5.896 | 6.310 |
| E | 6.094 | — | — |
| G | 6.832 | 7.050 | 7.300 |
| H | — | 6.084 | 6.470 |

These results indicate that the largest density of cells occurs when the broth is inoculated with the most cells. However, the differences in cell density are small. Growing the cells with double the concentration of solutions in the media resulted in the smallest cell density, possibly due to the higher concentration of salts in the media.

Lowering the secondary temperature did not result in a large difference in cell density.

Low densities were achieved when the cells were grown overnight at 37° C.

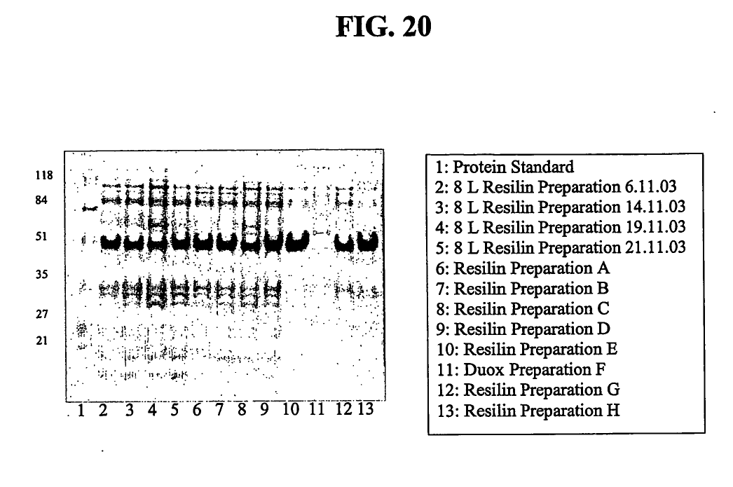

The cultures were spun, and processed through a Ni-NTA spin column. The elutions, with 1M Imidazole, were loaded onto an SDS PAGE gel. The results are shown in FIG. 20.

All the pellets were lysed in the same volume of lysis buffer and sonicated for the same amount of time. They were spun for 30 minutes at 14,000 rpm and 1 mL of the supernatant was loaded onto the Ni-NTA columns. The resilin was eluted with 100 μL 1 M Imidazole 5 μl of each eluate was loaded onto the gel.

The results show that the largest yield of resilin appears to be from the overnight cultures grown at 37° C. These samples were also shown to have the lowest optical density suggesting that although the yield of cells is lower, they contain more resilin protein.

The lowest yield appears to be from variation C which was inoculated with the largest amount of cells and reached the highest optical density. This suggests that the cells have used their energy to grow rather than produce resilin protein.

### Example 7

### Purification of Recombinant (E. coli) Hexahis Resilin

A summary of the procedure is as follows:
1. Lysis of Cells
2. Centrifugation
3. Pass supernatant through a Q-Sepharose column and collect the breakthrough.
4. Pass the breakthrough through a Ni NTA Column.
5. Elute the resilin with imidazole.
6. Concentrate Resilin
7. Dialysis
8. Concentrate through Amicon Ultra-15 (15 kDa cutoff) ultrafilter.
   1. Lysis of Cells

Thaw ˜100 g of cell pellet and resuspend in 400 ml of Lysis buffer. Place this material into 12×50 ml tubes and top up the tubes with the remainder of the lysis buffer. This helps to give a more even distribution of the cell paste into the 50 ml tubes. Each tube should contain about 40 ml of cell suspension.

### Sonication

An Ultrasonics (Melbourne, Aust) A180 (180 W max power output) sonicator with 10 mm ultrasonic probe was used for cell disruption. Place a 50 ml tube containing ca. 40 ml cell suspension into a beaker containing a wet ice slurry. Sonicate (for 30 sec). Sonicate the remainder of the material in the 12 tubes in turn, then repeat the procedure twice more. After each sonication, store the tubes in ice to enable the material to cool between sonications. When finished, place the tubes at −80° C. for at least 4 hours (or preferably overnight). It is easiest to place the tubes into a rack, place the rack into a polystyrene box and place this into the freezer.

Thaw the material by filling the polystyrene box with warm water. Sonicate as before for another 3×1 minute bursts. The cell suspension should now be a straw-coloured solution with no obvious viscosity (determined by dispensing an aliquot through a Pasteur pipette).

2. Centrifugation

Place the lysed cell suspension into Beckman thick walled polycarbonate tubes for spinning at 100,000 g at 4° C. for 30 minutes.

The supernatant should now be very clear and should not require filtering (except for the last few drops at the bottom of each Beckman tube). Collect the pellet into labelled containers and store in the −80° C. freezer.

3. Q-Sepharose Column (Anion Exchange) Flow-Through Chromatography)

Equilibrate the Q-sepharose column (˜200 ml lysis buffer for a 50 mm dia×100 mm resin column bed). Fluid can be run through the 50 mm dia column at a flow rate 10 ml per minute.

Once the column is equilibrated, load the supernatant onto the column at the same flow rate. Collect the breakthrough as this contains the resilin (pI=9.0). Begin collecting after ˜80 ml of supernatant has been loaded to ensure that all the resilin is collected.

Once loaded, use a small amount of lysis buffer to rinse the bottom of the supernatant container and load this onto the column. Continue washing with lysis buffer until all non-bound protein has passed through the column and the A280 has returned to baseline (˜280 ml required).

To the pooled breakthrough fluid add NaCl to 500 mM and Imidazole to 10 mM, adjust the pH to 8.0. Any resultant precipitate should be removed by either filtration or centrifugation.

To remove the proteins bound to the Q-sepharose column, elute with lysis buffer containing 1M NaCl. Once all the protein has been removed, re-equilibrate the column with ˜200 ml lysis buffer ready for the next run. The eluted protein can be discarded.

Immobilized metal affinity chromatography (Ni-NTA resin)

Assemble the Nickel column in a fume hood and equilibrate with wash buffer 1, (˜200 ml for a 50 mm dia×60 mm column). Flow rate ˜10 ml per minute.

Once the column is equilibrated, load the Q-sepharose breakthrough onto the column at the same flow rate. Collect the breakthrough, this will be discarded at a later point once it has been confirmed that it contains no resilin. Begin collecting after ˜40 ml of supernatant has been loaded.

Once loading is complete, use a small amount of wash buffer 1 to rinse the bottom of the Q-sepharose breakthrough container and load this onto the column. Continue washing the column with wash buffer 1 until the A280 has returned to baseline (˜100 ml). At this point, all the resilin should be bound to the nickel column and almost all other protein washed out and collected as Nickel column breakthrough.

Elution of Bound Resilin:

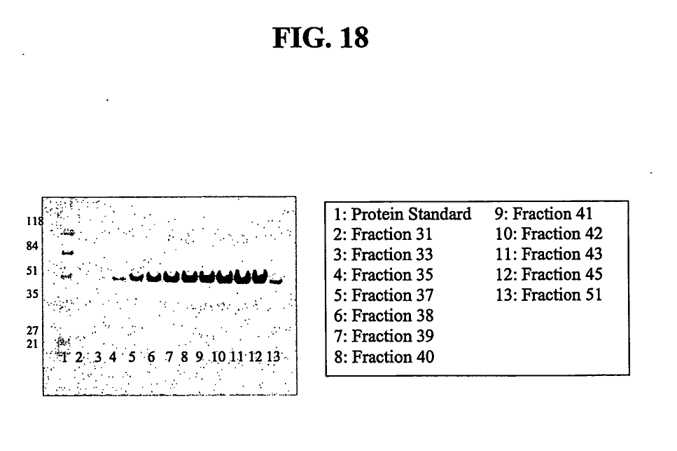

Connect the column to the FPLC and wash with 50 mM Imidazole solution (8.1% Elution Buffer, 91.9% Wash Buffer 2). Continue washing until OD baseline stabilises. Run a gradient from 8.1% Wash Buffer 2 (50 mM Imidazole) to 40% Wash Buffer 2 (200 mM Imidazole) over one hour at a flow rate of 5 ml/min. Collect 10 ml fractions. Continue eluting with 40% Wash Buffer 2 for another 50 minutes whilst collecting fractions. This should ensure that all the resilin and any other proteins have been removed from the nickel column. Label the fractions (FIG. 18) and store in the 4° C. fridge.

Re-equilibrate the nickel column with ˜200 ml wash buffer 1 ready for the next run.

Concentrate of Resilin

Concentrate the fractions containing resilin using a Millipore/Amicon ultra-filtration tube (cut off 10 kDa) to a final volume of −20 ml. Keep the flow through and check that it does not contain any resilin by running an SDS PAGE.

Dialyse and Concentrate

Dialyse the resilin using a 10 kDa cut off membrane, overnight against 5 litres of 50 mM Tris pH 7.5 and 50 mM NaCl.

Further concentrate the resilin to at least 200 mg/mL. At this point it should appear as a viscous yellow fluid at the bottom of the concentrating tube. The resilin is now ready to be used for experimentation.

### Buffers

Lysis Buffer:

50 mM TRIS

1 mM Benzamidine HCl

0.5% Triton X-100 (TX-100)

10 mM 1-ME (750 μl per litre of solution)

Make up to 1 litre with distilled water, pH to 7.2 (with conc HCl). Add the 2ME just prior to using and re-pH.

Wash Buffer 1:

100 mM NaH₂PO₄

10 mM TRIS

500 mM NaCl

1 mM Benzamidine HCl

10 mM Imidazole

0.1% Tx-100

10 mM 2ME (add 750 μl to 1 litre of solution just before using)

Make up to 1 litre with distilled water, pH to 8.0.

Wash Buffer 2 (Solution A)

100 mM Na₂PO₄

10 mM TRIS

500 mM NaCl

1 mM Benzamidine HCl

500 mM Imidazole

Make up to 1 litre with distilled water, pH to 7.2.

Elution Buffer (Solution B)

100 mM NaH₂PO₄

10 mM TRIS

500 mM NaCl

1 mM Benzamidine HCl

500 mM Imidazole

Make up to 1 litre with distilled water, pH to 7.2.

### Example 8

### Identification and Isolation of Resilin Homologues

A search of the genbank insect genomes database comprising completed genomes from Drosophila melanogaster, Anopheles gambiae and Apis mellifera (http://www.ncbi.nlm.nih.gov/BLAST/Genome/Insects.html) was carried out using the putative resilin gene (CG15920) from Drosophila (Ardell and Andersen, 2001) as the query sequence in a TBLASTN search using default settings and revealed a number of gene homologues with high scores (Low E values) all of which contain the “YGAP” amino acid motif. The repeat motif is of varying spacing and there are different numbers of repeat units in these genes. In Anopheles, only one sequence in the genome contains multiple YGAP repeat motifs (SEQ ID NO: 4), whereas in both Drosophila and Apis, there are two homologue forms (SEQ ID Nos: 5 and 6 and SEQ ID Nos: 6 and 7, respectively). These have similarity to the CG15920 type and the CG7709 type sequence.

Furthermore, Resilin homologues were isolated from insect cDNA in experiments employing degenerate oligonucleotide primers whose design was based on the alignment of primary amino acid sequences from Drosophila (CG15920) and Anopheles (EAA07479.1). This alignment is shown below. These degenerate oligos were used in PCR reactions with cDNA isolated from the pupal stages of fleas and buffalo flies. The sequence of primers is shown in the following Table.

| | Protein | |
|---|---|---|
| Name | sequence | Nucleotide sequence (5′-3′) |
| CF1 | GGNGG F′ | 5′ ggATAACAATTTCACACAgggg(inosine) |
| | | gg(inosine)AAYgg(inosine)gg |
| | | (inosine)Mg 3′ |
| CF2 | GNGNG F′ | 5′ ggATAACAATTTCACACAgggg(inosine)AAY |
| | | gg(inosine)AAYgg 3′ |
| CF3 | YGAP F′ | 5′ ggATAACAATTTCACACAggTAYgg(inosine) |
| | | gC(inosine)CC 3′ |
| CF4 | GNGNG R′ | 5′ CACgACgTTgTAAAACgACCCRTT(inosine)C |
| | | CRTT(inosine)CC 3′ |
| CF5 | YGAP R′ | 5′ CACgACgTTgTAAAACgACgg(inosine)gC |
| | | (inosine)CCRTA 3′ |
| CF6 | SYGAP F′ | 5′ ggATAACAATTTCACACAggCC(inosine)SW |
| | | (inosine)SWRTA(inosine)CC 3′ |
| CF7 | GYSSG R′ | 5′ ggATAACAATTTCACACAggWS(inosine)TAY |
| | | gg(inosine)gC(inosine)CC 3′ |

Degenerate Primers Designed and Used in this Experiment

1. PCR Experiments (Optimization of PCR Conditions And MgCl₂ Concentration)

PCR's were set up to determine the optimal conditions for amplification of specific products from the primer pairs designed (see table of primer pairs above). The standard PCR was set up as follows by adding all components listed below in to a microcentrifuge PCR tube to a total volume of 50 μl: (note that QIAGEN Taq Polymerase kit was used)

10×QIAGEN reaction buffer 5 μl, 5×Q buffer 10 μl, 25 mM MgCl₂ (variable component ranging from 0.2 μl-2 μl), dNTP mix (0.5 μM each) 0.5 μl, primer F′ 0.5 μl, primer R′ 0.5 μl, Taq polymerase 0.5 μl, sterile water) (variable 31.8-30 μl), template DNA 1 μl.

Conditions used for the PCR was variable as well (machine used BIORAD “Gene Cycler”):

94° C. 30 sec

37° C. 30 sec (variable step)

72° C. 1 minute

for 35 cycles (variable step)

cycles testing included 40 cycles and annealing temperatures tested included 40° C., 47° C.

other conditions tested include two stage PCR:

94° C. 30 sec

37° C. 30 sec

72° C. 1 minute

for 5 cycles

94° C. 30 sec

66° C. 30 sec

72° C. 1 minute

for 40 cycles

2. Cloning of PCR Product in pGEM-Teasy™

Run PCR products on a medium size agarose gel (120 ml+1.2 μl EtBr) and excise bands after run with fresh scalper blade. Place cut agarose into 2 ml microcentrifuge tubes. Purify using the Macherey-Nagel Nucleospin extract 2 in 1 kit (protocol 4.1: protocol for DNA extraction from agarose gels):

For each 100 mg of agarose gel, add 300 μl buffer NT1. Incubate sample at 50oC for 5-10 minutes with brief vortexing every 3 minutes until totally dissolved. Then place NucleoSpin Extract column into a 2 ml collecting tube, load sample and centrifuge 1 minute at 8,000×g (10,000 rpm). Then an optional step can be performed by discarding the flowthrough and placing the column back into the collecting tube and adding 500 μl buffer NT2. centrifuge for 1 minute at full speed. Discard flowthrough and place back into collecting tube. Add 600 μl of buffer NT3 and centrifuge for 1 minute at full speed. Discard flowthrough and place back into collecting tube. Then add 200 μl buffer NT3. Centrifuge for 2 minutes at full speed to remove NT3 quantitatively. Finally place column into a clean 1.5 ml microcentrifuge tube and add 25-50 μl elution buffer NE and leave at room temperature for 1 minute. Centrifuge for 1 minute at full speed.

Ligate excised PCR fragment into pGEM-Teasy (Promega) by putting together:

PCR fragment 3.7 μl, pGEM-Teasy 0.5 μl, ligation buffer 5 μl and T4 DNA ligase 0.8 μl

And incubate overnight at 4oC for maximum transformants (can also be done for ½ hour at room temperature) and proceed to transformation protocol.

3. Transformation Protocol

Thaw top10 cells on ice and when thawed add 0.5 μl of res5 plasmid or 1 μl of ligation reaction. Mix gently with fingers and keep on ice for 1 hour. Heat shock tube at 42 degrees for 30 seconds, and immediately place on ice for 10 minutes. Add 250 μl of SOC and incubate for 1 hour at 37° C. Plate out at 25, 50 and 100 μl onto LB/amp plates with 3.5 μl of 1M IPTG and 16 μl of 50 ng/ml×Gal. Inoculate overnight at 37° C. Pick white colonies the next day and inoculate into LB/amp culture. Use the 15 ml blue capped Falcon tubes with 10 ml's of LB (10 μl of ampicillin to 10 mls of LB). Proceed to mini prep protocol (QIAGEN)

4. Mini Prep Protocol (QIAGEN)

Transfer 2 mls of culture from a 15 ml Blue Cap Falcon tube into a 2 ml microcentrifuge tube and spin for 10 minutes at max speed. Then decant supernatant into a glass beaker containing bleach and resuspend bacterial pellet in 250 μl buffer P1 via vortexing. Add 250 μl buffer P2 to resuspended cells and gently invert the tube 4 to 6 times to mix. Following that add 350 μl buffer N3 and invert the tube immediately 4 to 6 times. Centrifuge tubes for 10 minutes at maximum speed. A compact white pellet will form. Using a pipette, transfer the supernatant to a QIAprep column and centrifuge 30 to 60 seconds. Discard the flow-through. An optional step after this is to wash the column by adding 0.5 ml buffer PB and centrifuge 30 to 60 seconds. Discard the flow through from this and wash the column by adding 0.75 ml buffer PE and centrifuge for 30 to 60 seconds. Discard the flow-through from this and centrifuge an additional 1 minute to remove residual wash tube. To elute the DNA, add 50 μl buffer EB to the centre of the column and let stand for 1 minute. Then centrifuge for 1 minute.

5. Sequencing Protocol

Put together in a microcentrifuge tube:

Double distilled water 1 μl, DNA (plasmid) 5 μl, primer (M13 F′, M13 R′ or T7) 1 μl, Big Dye 3.1 2 μl, sequencing buffer 341 (total volume 12 μl) and use program 4 35 cycles. When complete, add 1.3 μl 3M NaOAc pH5.2 and 30 μl absolute ethanol. Incubate at −20° C. for 15 minutes. Spin 15 minutes at 4° C. and remove solution carefully by pipetting. Then wash with 100 μl 80% ethanol and spin at max speed for 5 minutes at 4° C. Then remove solution carefully and dry with no heat in vacuum centrifuge for 3 minutes. Make sure that the sequencing cleanup is performed in 1.5 ml microcentrifuge tubes. Also better sequences were obtained when the amount of starting DNA was increased from 5 μl to 6 μl.

6. RNA Extraction with QIAGEN Rneasy Mini Kit Following Protocol Described in “Rneasy Mini Protocol for Isolation of Total RNA from Animal Tissue”

Tissues and samples need to be disrupted first up. To do this the samples are first places in a sterile RNase free 2 ml screw cap microcentrifuge tube with 3 to 4 sterile glass beads. This is then taken through the BIO-101 (Savant) FastPrep FP120 disrupter. A speed of 5.0 and time of 3×6 seconds is used. The a quick spin for 15 seconds at 2000 rpm is performed to allow settling of debris. The supernatant is then transferred onto a QIA shredder column in a 2 ml collection tube and then centrifuged for 15 seconds at 10,000 rpm. The cleared lysate is then transferred into a fresh 1.5 ml microcentrifuge tube and further centrifuge for an extra 3 minutes at max speed. This is then transferred into another fresh microcentrifuge tube. Then 1 volume (approximately 350-600 μl) of 70% ethanol is added to the cleared lysate and mixed immediately by pipetting (do not centrifuge). Up to 700 μl of the sample can then be added to the Rneasy column, placed in a 2 ml collection tube. Centrifuge for 15 seconds at 8000×g (10,000 rpm). Discard the flow through and pipette 350 μl buffer RW1 onto the column and centrifuge 15 seconds at 8000×g (10,000 rpm). Discard the flow through and add 10 μl Dnase 1 stock solution to 70 μl buffer RDD. Mix this by gentle inversion. Pipette Dnase 1 incubating mix (80 μl) directly onto Rneasy silica-gel membrane and place on bench top (20-30oC) for 15 minutes. Pipette 350 μl buffer RW1 onto column and centrifuge 15 seconds at 8000×g. discard flow-through and then add 700 μl buffer RW1 to column and centrifuge 15 seconds at 8000×g. discard flow-through and collecting tube. Then transfer Rneasy column into a new 2 ml collection tube and pipette 500 μl buffer RPE onto the column. Centrifuge for 15 seconds at 8000×g and discard flow-through. Add another 500 μl buffer RPE to the column and centrifuge 2 minutes at 8000×g to dry membrane. Place Rneasy column into a new 2 ml collecting tube and centrifuge at max speed for 1 minute. To elute, place the column in a new 1.5 ml microcentrifuge tube and pipette 30-50 μl Rnase-free water directly onto the column and then centrifuge for 1 minute at 8000×g.

7. Superscript Double Stranded cDNA Synthesis Kit (Invitrogen)

1^{st} Strand Synthesis

Add into a RNase free 1.5 microcentrifuge tube:

primer (100 μmol/μl) 1 μl, RNA in DEPC-treated water 11 μl

and heat mix to 70° C. for 10 minutes and quick chill on ice. Collect contents at the bottom of the tube by brief centrifugation and add:

5× first strand reaction buffer 411, 0.1M DTT 2 μl, 10 mM dNTP mix 1 μl

Vortex gently and collect by brief centrifugation. Place tube at 45° C. for 2 minutes and add superscript II RT 1 μl.

Mix gently and incubate at 45° C. for 1 hour. Total volume is now 20 μl. Then place tube on ice to terminate reaction

2^{nd} Strand cDNA Synthesis

On ice add the following components to the first strand reaction tube:

DEPC-treated water 91 μl, 5× second strand reaction buffer 30 μl, 10 mM dNTP mix 311, E. coli DNA ligase (10 U/μl) 1 μl, E. coli DNA Polymerase I (10 U/μl) 4 μl, E. coli Rnase H (2 U/μl) 1 μl. Vortex gently to mix and incubate 2 hours at 16° C. (temperature must not exceed 16° C.). Then add 2 μl (10 units) of T4 DNA polymerase and continue to incubate at 16° C. for 5 minutes. Place tube on ice and add 10 μl of 0.5M EDTA and add 160 μl of phenol:chloroform:isoamyl alcohol (25:24:1), vortex thoroughly and centrifuge at room temperature for 5 minutes at 14,000×g. Carefully remove 140 μl of upper, aqueous layer and transfer to a fresh 1.5-ml tube. Add 70 μl of 7.5M NH₄Oac, followed by 0.5 ml of ice-cold absolute ethanol. Vortex the mixture thoroughly and immediately centrifuge at room temperature for 20 minutes at 14,000×g. Remove supernatant carefully and discard. Overlay the pellet with 0.5 ml ice-cold 70% ethanol. Centrifuge for 2 minutes at 14,000×g and remove supernatant and discard. Finally dry the pellet at 37° C. for 10 minutes to evaporate residual ethanol and dissolve pellet in a small volume of DEPC-treated water (3 μl per 25 μg of starting total RNA or 1 μg of starting mRNA).

Results from Degenerate PCR

Initial optimization experiments were performed with the res5 plasmid and primer pair's 1+5, 2+5, 1+4 and 3+4. Conditions used were as described 1. PCR experiments (optimization of PCR conditions and MgCl₂ concentration). Of all the conditions tested, the optimal condition was found to be at 37° C. for 35 cycles. PCR was done in the BIORAD “gene cycler” PCR machine using QIAGEN reagents. The optimal MgCl₂ concentration was found to be 0.5 μl. A higher MgCl₂ concentration resulted in smearing. Optimization experiments showed that the use of Q buffer improved the efficacy of the reaction resulting in brighter and sharper bands.

The next stage involved extracting RNA and making ds cDNA from flea and buffalo fly this was then used in degenerate PCR. Also at this stage, two new degenerate primers were designed, primers 6 and 7. This primers were used in conjunction with the earlier primers. PCR was then performed using all the primer pair's 1+7, 2+7, 3+7 and the earlier primer sets of 1+5, 2+5, 1+4 and 3+4 on both flea and buffalo fly cDNA. Of these bands were obtained for buffalo fly for primer pair's 1+7 (approx. 500 bp), 2+7 (300, 500 bp and 1 kb), 3+7 (1 kb), 1+4 (approx. 1 kb) and 3+4 (approx. 1 kb) (see FIG. 36). Bands were obtained in flea for primer pair 2+5 300 and 500 bp) (FIG. 37).

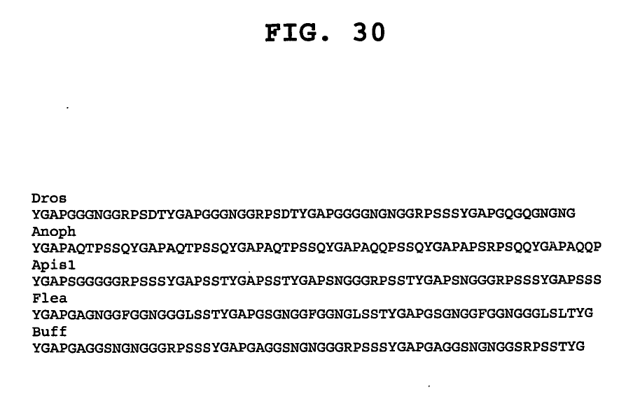

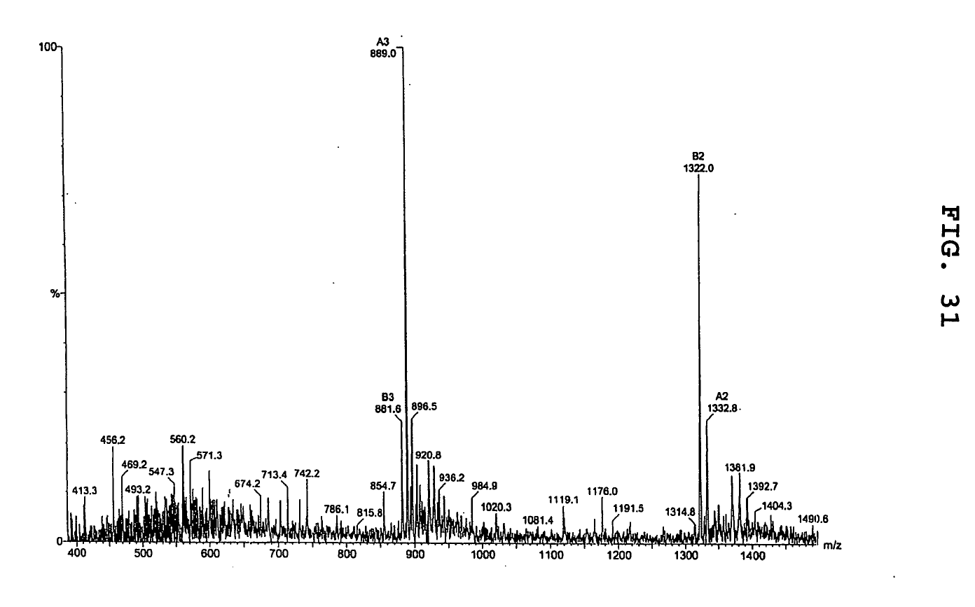

Partial nucleotide sequences were then obtained via cloning of these bands from flea (SEQ ID NOs: 8, 9 and 12) and buffalo fly (SEQ ID NOs: 10, 11 and 13). When translated, these sequences showed the repeat motif YGAP as seen in FIG. 30. FIG. 30 also illustrates the similarities (and differences) between sequences containing the repeat motifs.

### Example 9

### Synthetic Resilin Production

Primers were designed based upon the sequence of the putative structural domain of Anopheles and Drosophila resilin genes (or homologues), such that they could be ligated to form contamers of the structural motif.

| | |
|---|---|
| AnSyn1 | 5′ pGCGCAAACCCCGTCTAGCCAGTATGGTGCACCG 3′ |
| | \|\|\|\|\|\|\|\|\|\|\|\|\|\|\|\|\|\|\|\|\|\|\|\|\|\|\|\| |
| Ansyn2 | 3′ TTGGGGCAGATCGGTCATACCACGTGGCCGCGTp |
| Drosyn1 | 5′ pCGTCCGAGCGATACCTATGGTGCGCCGGGTGGTGGCAACGGTGGT 3′ |
| | \|\|\|\|\|\|\|\|\|\|\|\|\|\|\|\|\|\|\|\|\|\|\|\|\|\|\|\|\|\|\|\|\|\|\|\|\|\|\|\|\| |
| Drosyn2 | GCTCGCTATGGATACCACGCGGCCCACCACCGTTGCCACCAGCAGp |

### Annealing and Ligation

10 ul 10× Ligation Buffer (New England Biolabs)

10 ul 100 uM AnSyn1 or DroSyn1

10 ul 100 uM AnSyn2 or DroSyn2

69 ul dH20

100 ul final volume

Annealing was carried out by an incremental decrease in temperature from 50 to 10° C. over minutes (0.5° C./min) on a MJ Research PTC-200 Peltier Thermal cycler.

Follow annealing 400 U of T4 Ligase (New England Biolabs) was added to each tube, and ligation performed at 16° C. for 1-18 hrs. Samples were heated at 65° C. for 10 minutes to inactivate Ligase, and samples purified using Qiaquick DNA purification columns (Qiagen).

### End-Filling Reactions

5 ul 10×ExoPol Buffer (New England Biolabs)

5 ul 10 mM dNTPs

39 ul Template (annealed and ligated DroSyn or AnSyn primer concatamers)

1 ul (5 U) Klenow fragment (New England Biolabs)

50 ul final volume

were prepared on ice. Reactions were incubated at 25° C. for 15 minutes, then stopped by adding 1 ul 0.5 M EDTA

and heating at 75° C. for 20 minutes.

Initial cloning was performed using the ZeroBlunt TOPO cloning vector (Invitrogen), with transformation in supercompetent SURE2 cells (Stratagene). Selection of recombinants was based upon growth on LB agar plates containing Kanamycin at 25 ug/ml, followed by PCR screening of selected colonies using M13for and M1rev primers to amplify inserts within the plasmids. Due to difficulties in obtaining clones without sequence mutation especially on the DroSyn reactions, attributed to toxicity of expressed products, different cell lines including Stb12, Stb13 and M15-[pREP4] have all been used.

AnSyn Clone Isolation:

Initially 12 recombinants containing AnSyn products in the size range 200-500 bp were selected for sequencing. Sequencing was carried out using BigDye 3.1 chemistry (Applied Biosystems) and either M13for or M13rev primers. All were found to contain mutations (insertions or deletions). Six contained insertions or deletions within the sequence, and were removed. The other 6 all had truncated 5′ sequences such that the desired open reading frame (ORF) was disrupted. However despite this truncation, they were otherwise perfect concatamers ranging from 7-13 repeats in length. Below are examples of the sequences obtained which have been allocated SEQ ID Nos: 17-24 in the attached sequence listing. Also below is the amino acid sequence (SEQ ID NO:25) of the polypeptide encoded by SEQ ID NO:24.

Clone 1 comprises SEQ ID NO: 17 which is well expressed except that it is eaten at 5′ end, and therefore has only 10 repeats:

AACCCCGTCTAGCCAGTATGGTGCACCG
GCGCAAACCCCGTCTAGCCAGTATGGTGCACCG
GCGCAAACCCCGTCTAGCCAGTATGGTGCACCG
GCGCAAACCCCGTCTAGCCAGTATGGTGCACCG
GCGCAAACCCCGTCTAGCCAGTATGGTGCACCG
GCGCAAACCCCGTCTAGCCAGTATGGTGCACCG
GCGCAAACCCCGTCTAGCCAGTATGGTGCACCG
GCGCAAACCCCGTCTAGCCAGTATGGTGCACCG
GCGCAAACCCCGTCTAGCCAGTATGGTGCACCG
GCGCAAACCCCGTCTAGCCAGTATGGTGCACCGGCGC

Clone 9 comprises SEQ ID NO: 18 includes an inserted C near the start and is not preferred:

GCGCCAAACCCCGTCTAGCCAGTATGGTGCACCG
GCGC AAACCCCGTCTAGCCAGTATGGTGCACCG
GCGC AAACCCCGTCTAGCCAGTATGGTGCACCG
GCGC AAACCCCGTCTAGCCAGTATGGTGCACCG
GCGC AAACCCCGTCTAGCCAGTATGGTGCACCG
GCGC AAACCCCGTCTAGCCAGTATGGTGCACCG
GCGC AAACCCCGTCTAGCCAGTATGGTGCACCG
GCGC AAAaCCCGTctAGCCAgTATGGTGCACCGGCG

Clone 10 comprises SEQ ID NO: 19 which is well expressed except that it is eaten at 5′ end, and therefore has only 12 repeats:

GCAAACCCCGTCTAGCCAGTATGGTGCACCG
GCGCAAACCCCGTCTAGCCAGTATGGTGCACCG
GCGCAAACCCCGTCTAGCCAGTATGGTGCACCG
GCGCAAACCCCGTCTAGCCAGTATGGTGCACCG
GCGCAAACCCCGTCTAGCCAGTATGGTGCACCG
GCGCAAACCCCGTCTAGCCAGTATGGTGCACCG
GCGCAAACCCCGTCTAGCCAGTATGGTGCACCG
GCGCAAACCCCGTCTAGCCAGTATGGTGCACCG
GCGCAAACCCCGTCTAGCCAGTATGGTGCACCG
GCGCAAACCCCGTCTAGCCAGTATGGTGCACCG
GCGCAAACCCCGTCTAGCCAGTATGGTGCACCG
GCGCAAACCCCGTCTAGCCAGTATGGTGCACCGGCGC

Clone 15 comprises SEQ ID NO: 20 which is well expressed except that it is eaten at 5′ end, and therefore has only 10 repeats:

AACCCCGTCTAGCCAGTATGGTGCACCG
GCGCAAACCCCGTCTAGCCAGTATGGTGCACCG
GCGCAAACCCCGTCTAGCCAGTATGGTGCACCG
GCGCAAACCCCGTCTAGCCAGTATGGTGCACCG
GCGCAAACCCCGTCTAGCCAGTATGGTGCACCG
GCGCAAACCCCGTCTAGCCAGTATGGTGCACCG
GCGCAAACCCCGTCTAGCCAGTATGGTGCACCG
GCGCAAACCCCGTCTAGCCAGTATGGTGCACCG
GCGCAAACCCCGTCTAGCCAGTATGGTGCACCG
GCGCAAACCCCGTCTAGCCAGTATGGTGCACCGGCGC

Clone 16 comprises SEQ ID NO: 21 which is well expressed except that it is eaten at 5′ end, and therefore has only 13 repeats

GCGCAAACCCCGTCTAGCCAGTATGGTGCACCG
GCGCAAACCCCGTCTAGCCAGTATGGTGCACCG
GCGCAAACCCCGTCTAGCCAGTATGGTGCACCG
GCGCAAACCCCGTCTAGCCAGTATGGTGCACCG
GCGCAAACCCCGTCTAGCCAGTATGGTGCACCG
GCGCAAACCCCGTCTAGCCAGTATGGTGCACCG
GCGCAAACCCCGTCTAGCCAGTATGGTGCACCG
GCGCAAACCCCGTCTAGCCAGTATGGTGCACCG
GCGCAAACCCCGTCTAGCCAGTATGGTGCACCG
GCGCAAACCCCGTCTAGCCAGTATGGTGCACCG
GCGCAAACCCCGTCTAGCCAGTATGGTGCACCG
GCGCAAACCCCGTCTAGCCAGTATGGTGCACCG
GCGCAAACCCCGTCTAGCCAGTATGGTGCACCGGCGC

Clone 18 comprises SEQ ID NO: 22 which is well expressed except that it is eaten at 5′ end, and therefore has only 7 repeats:

GCGCAAACCCCGTCTAGCCAGTATGGTGCACCG
GCGCAAACCCCGTCTAGCCAGTATGGTGCACCG
GCGCAAACCCCGTCTAGCCAGTATGGTGCACCG
GCGCAAACCCCGTCTAGCCAGTATGGTGCACCG
GCGCAAACCCCGTCTAGCCAGTATGGTGCACCG
GCGCAAACCCCGTCTAGCCAGTATGGTGCACCG
GCGCAAACCCCGTCTAGCCAGTATGGTGCACCGGCGC

Clone 22 comprises SEQ ID NO: 23 has an inserted A and is not preferred:

GCGCAAACCCCGTCTAGCCAGTATGGTGCACCG
GCGCAAACCCCGTCTAGCCAGTATGGTGCACCG
GCGCAAACCCCGTCTAGCCAGTATGGTGCACCG
GCGCAAACCCCGTCTAGCCAGTATGGTGCACCG
GCGCAAACCCCGTCTAGCCAGTATGGTGCACCG
GCGCAAACCCCGTCTAGCCAGTATGGTGCACCG
GCGCAAACCCCGTCTAGCCAGTATGGTGCACCG
GCGCAAACCCCGTCTAGCCAGTATGGTGCACCG
GCGCAAAACCCCGTCTAGCCAGTATGGTGCACCG
GCGCAAACCCCGTCTAGCAGTATGGTGCACCGG
CGCAAACCCCGTCTAGCCAGTATGGTGCACCGG
CGCAAACCCCGTCTAGCCAGTATGGTGCACCGG
CGCAAACCCCGTCTAGCCAGTATGGTGCACCGG
CGCAAACCCCGTCTAGCCAGTATGGTGCACCGG
CGCAAACCCCSTCWAGCCAGWATGGTGCACCGGCGC

Annealing and ligation was repeated as above following which the end-filling reaction was carried out using the proofreading DNA polymerase PfuUltra™ (Stratagene) as follows:

5 ul 10×PfuUltra HF reaction buffer (Stratagene)

4 ul 10 mM dNTPs

40 ul Template (annealed and ligated AnSyn primer concatamers)

1 ul (2.5 U) PfuUltra DNA polymerase (Stratagene)

50 ul final volume

This reaction was incubated at 72oC for 30 minutes.

Following incubation, the entire reaction was ran on a 1% agarose gel. Regions of the gel containing concatamers in the size ranges 400-500 bp, 800-1200 bp and 1650 bp+were excised, and DNA purified from gels using the QIAquick gel extraction kit (QIAGEN). The large fragments (1650 bp+) were ligated into pCR-Blunt and transformed into Stb13 cells. Ninety-six colonies were screened, resulting in 3 potential clones. These were sequenced. one (AnL 7H) was contained sequence corresponding to a concatamer of 19 repeats.

(SEQ ID NOs:24 and 25)

| | |
|---|---|
| An-L 7H clone | CAAACCCCGTCTAGCCAGTATGGTGCACCG |
| | GCGCAAACCCCGTCTAGCCAGTATGGTGCACCG |
| | GCGCAAACCCCGTCTAGCCAGTATGGTGCACCG |
| | GCGCAAACCCCGTCTAGCCMGTATGGTGCACCG |
| | GCGCAAACCCCGTCTAGCCAGTATGGTGCACCG |
| | GCGCAAACCCCGTCTAGCCAGTATGGTGCACCG |
| | GCGCAAACCCCGTCTAGCCAGTATGGTGCACCG |
| | GCGCAAACCCCGTCTAGCCAGTATGGTGCACCG |
| | GCGCAAACCCCGTCTAGCCAGTATGGTGCACCG |
| | GCGCAAACCCCGTCTAGCCAGTATGGTGCACCG |
| | GCGCAAACCCCGTCTAGCCAGTATGGTGCACCG |
| | GCGCAAACCCCGTCTAGCCAGTATGGTGCACCG |
| | GCGCAAACCCCGTCTAGCCAGTATGGTGCACCG |
| | GCGCAAACCCCGTCTAGCCAGTATGGTGCACCG |
| | GCGCAAACCCCGTCTAGCCAGTATGGTGCACCG |
| | GCGCAAACCCCGTCTAGCCAGTATGGTGCACCG |
| | GCGYAAACCCCGTCTAGCCAGTATGGTGCACCG |
| | GCGCAAACCCCGTCTAGCYAGTATGGTGCACC |

QTPSSQYGAP AQTPSSQYGAP AQTPSSQYGAP AQTPSSQYGAP
AQTPSSQYGAP AQTPSSQYGAP AQTPSSQYGAP AQTPSSQYGAP
AQTPSSQYGAP AQTPSSQYGAP AQTPSSQYGAP AQTPSSQYGAP
AQTPSSQYGAP AQTPSSQYGAP AQTPSSQYGAP AQTPSSQYGAP
AQTPSSQYGAP AQTPSSQYGAP AQTPSSQYGA

These clone are being re-amplified with specific primers and cloned into the expression vector as discussed below.

DroSyn Clone Isolation

Six DroSyn recombinants initially screened were deemed unacceptable due to the presence of mutations within the sequence such that the desired ORF was disrupted. It seemed that this high error rate was evidence that the constructs when expressed even at low levels proved toxic to the host bacterium, such that only mutated clones were being selected. In an attempt to overcome this problem different host cell types with tighter regulation of expression were chosen for future cloning experiments.

Screening of 96 colonies from a subsequent ligation of DroSyn end-filled product transformed into Stb12 cells (Invitrogen) proved unsuccessful also. Six recombinants were selected and sequenced but all were inappropriate to indels. Subsequently 19 further clones obtained following ligation and transformation into Stb13 cells (Invitrogen) were also isolated and characterised. Seventeen were inappropriate due to introduced sequence errors, however 1 cloned containing 4 repeats were obtained. Although small, these are currently being re-amplified with specific primers and cloned into the expression vector as discussed below.

(SEQ ID NOs: 26 and 27)

K3F
GTCCGAGCGATACCTATGGTGCGCCGGGTGGTGGCAACGGTGGTCGTCCG
AGCGATACCTATGGTGCGCCGGGTGGTGGCAACGGTGGTCGTCCGAGCGA
TACCTATGGTGCGCCGGGTGGTGGCAACGGTGGTCGTCCGAGCGATACCT
ATGGTGCGCCGGGTGGTGGCAACGGTGG

AA = RPSDTYGAPGGGNGG RPSDTYGAPGGGNGG RPSDTYGAPGGGNGG RPSDTYGAPGGGNG

This clone is being re-amplified with specific primers and cloned into the expression vector as discussed below.

Re-Amplification for Cloning into Expression Vector Pet

Primers were designed for the re-amplification of selected inserts such that a Nde I restriction enzyme site and 6-HIS expression tag were added to the 5′ end of the concatamer, while a battery of stop codons and EcoR1 restriction enzyme site were added at the 3′ end. These were designed such that the amplified product could be cloned into the expression vector pET using the EcoR1 and Nde I restriction enzymes such that the product when expressed would contain an N-terminal 6-histidine tag for easy purification of the expressed protein.

Addition of 8 bases of AnSyn or DroSyn specific sequence at the 5′ end of the primers enabled the re-introduction of ORF in those previously discussed constructs containing small 5′ deletions. To facilitate correct cleavage of the recombinant clone with EcoR1 and Nde1, the pCR-BLUNT-specific sequence within the primers has been modified with the replacement of an A with a T (shown in lowercase) to remove the EcoR1 sites normally present at these sites.

```
pET-Eco (mod EcoR1 in plasmid)
        Eco STOPS pCR-BLUNT VECTOR SEQ
GCGAATTCTCACCTAAATATCTGCAGtATTCAGG
pET-NdeAn
      Nde His-6 tag pCR-BLUNT VECTOR AnSyn
seq
GCCATATGCATCACCATCACCATCACGCCAGTGTGCTGGtATTCAGGGCG
CAAAC
pET-NdeDros
      Nde His-6 tag pCR-BLUNT VECTOR
DroSyn seq
GCCATATGCATCACCATCACCATCACGCCAGTGTGCTGGtATTCAGGCGT
CCGAG
```

Initially clones 10 and 16 from above were used to optimise amplification using the primers above.

Expand High Fidelity PCR system (Roche) was used to amplify the clones using the following protocol.

Per reaction, 4 ul 10 mM dNTPs
1 ul 10 uM pET-NdeAn primer
1 ul 10 uM pET-Eco primer
17 ul dH20

2 ul 1/10 dilutes An16 or An10 plasmid

To each tube add 25 ul of master mix containing:

2 ul 10× Expand High Fidelity Reaction buffer 2

0.75 ul HF enzyme mix

22.25 ul dH₂O

PCR was performed under the following conditions:

Denature 95° C. for 2 min

| | | |
|---|---|---|
| Cycle 1 | 95° C. for 30 sec | |
| | 50° C. for 30 sec | |
| | 72° C. for 2 min | Repeat cycle 4 times |
| Cycle 2 | 95° C. for 30 sec | |
| | 64° C. for 30 sec | |
| | 72° C. for 2 min | Repeat cycle 19 times |
| | 72° C. for 10 min | |

The PCR reactions were run on a 1% agarose gel and products were purified from gel using a QIA quick gel extraction kit (QIAGEN).

Products were cloned using the pCR-Blunt cloning vector (Invitrogen), with transformation into chemically competent Stb13 cells (Invitrogen). Selection of recombinants was based upon growth on LB agar plates containing Kanamycin at 50 ug/ml, followed by PCR screening of selected colonies using M13for and M1rev primers to amplify inserts within the plasmids.

Selected recombinants were sequenced to confirm the correct sequence had been cloned including the presence of Nde1 and EcoR1 restriction enzyme digestion sites, 6-His tag and stop codons.

Result: A number of correct clones were isolated two of which were chosen for further study. Below is their sequence (SEQ ID NO: 28 and 30) and predicted protein sequence (SEQ ID NO: 29 and 31).

T2A - 7 repeats
GCCATATGCATCACCATCACCATCACGCCAGTGTGCTGGTATTCAGGG
CGCAAACCCCGTCTAGCCAGTATGGTGCACCGGCGCAAACCCCGTCTAGCCAGTATGG
TGCACCGGCGCAAACCCCGTCTAGCCAGTATGGTGCACCGGCGCAAACCCCGTCTAGC
CAGTATGGTGCACCGGCGCAAACCCCGTCTAGCCAGTATGGTGCACCGGCGCAAACCC
CGTCTAGCCAGTATGGTGCACCGGCGCAAACCCCGTCTAgCCAGTATGGTGCACCGGC
GCCCTGAATACTGCAGATATTTAGGTGAGAATTCGC
H Met H H H H H H A S V L V F R
A Q T P S S Q Y G A P
A Q T P S S Q Y G A P
A Q T P S S Q Y G A P
A Q T P S S Q Y G A P
A Q T P S S Q Y G A P
A Q T P S S Q Y G A P
A Q T P S S Q Y G A P A P Stop I L Q I F R Stop E F
R5E - 10 repeats
GCCATATGCATCAccAtCAcCATCACgCCAGTGTGCtgGTATTCAGGGCGC
AAACCCCGTcTAGCCAGTATGGTGCaCCGGCGCAAACCCcGTcTAGCCAGTATGGTGC
ACCGGCGCAAACCCCGTCTAGCCAGTATGGTGCACCGGCGCAAACCCCGTCTAGCCAG
CTAgCCAGTATGGTGCACCGGCGCAAACCCCGTCTAGCCAGTATGGTGCACCGGCGCA
AACCCCGTCTAGCCAGTATGGTGCACCGGCGCAAACCCCGTCTAGCCAGTATGGTGCA
CCGGCGCAAACCCCGTCTAGCCAGTATGGTGCACCGGCGCCCTGAATACTGCAGATAT
TTAGGTGAGAATTCGC
H Met H H H H H H A S V L V F R
A Q T P S S Q Y G A P
A Q T P S S Q Y G A P
A Q T P S S Q Y G A P
A Q T P S S Q Y G A P
A Q T P S S Q Y G V P
A Q T P S S Q Y G A P
A Q T P S S Q Y G A P
A Q T P S S Q Y G A P
A Q T P S S Q Y G A P
A Q T P S S Q Y G A P A P Stop I L Q I F R Stop E F

The T2A and R5E inserts have been cut out of the pCR-Blunt vector in the following manner.

| | |
|---|---|
| 5 ul | Plasmid |
| 1 ul | Nde I enzyme (Mew England Biolabs) |
| (20 U) | |
| 1 ul | 10× Buffer 4 (New England Biolabs) |
| 3 ul | dH2O |
| 20 ul | |

The reaction was incubated at 37° C. for 2 hours, then 1 ul (10 U) EcoR1 enzyme (New England Biolabs) was added. The reaction was incubated for a further 2 hours.

The reactions were run on a 1% agarose gel and digested T2A and R5E products were purified from gel using a QIA quick gel extraction kit (QIAGEN).

The pET expression vector containing a 500 bp DHFR insert (RSC722+DHFR) was obtained from Dr Nick Dixon—(RSC, ANU; Neylon et al. 2000). This was cloned into competent DH5□ cells. Colonies were selected and grown up at 37° C. overnight, following which plasmids were isolated using a plasmid purification kit (QIAGEN). The insert was cut out by the following protocol.

| | |
|---|---|
| 5 ul | Plasmid |
| 1 ul | Nde I enzyme (New England Biolabs) |
| (20 U) | |
| 1 ul | 10× Buffer 4 (New England Biolabs) |
| 3 ul | dH2O |
| 20 ul | |

The reaction was incubated at 37oC for 2 hours, then 1 ul (10 U) EcoR1 enzyme (New England Biolabs) was added. The reaction was incubated overnight.

The reactions were run on a 1% agarose gel and the digested plasmid was purified from gel using a QIA quick gel extraction kit (QIAGEN).

Ligations into expression vector:

Ligations were carried out as follows:

| | |
|---|---|
| 10 ul | plasmid |
| 7 ul | insert |
| 2 ul | 10× ligation buffer (New England Biolabs) |
| 1 ul | T4 ligase (New England Biolabs) |
| (400 U) | |
| 7 ul | dH2O |
| 20 ul | |

Incubated overnight at 4° C.

Transformed into chemically competent M15 [pREP4] cells, and selected on agar plates containing 100 ug/ml Ampicillin and 25 ug/ml kanamycin.

Integrity of sequence will be confirmed by sequence analysis, after which these plasmids will be transformed into appropriate cells for expression.

### Example 10

A peptide was synthesised using standard FMOC synthesis techniques. The peptide has the following amino acid sequence (SEQ ID NO:32):

RPSDTYGAPGGGNGGRPSDTYGAPGG (26mer)

The peptide includes a 15-residue repeat sequence and spans two tyrosine residues.

There are two alternative synthesis for the peptide.

Strategy 1:

1. Use Wang-Gly resin: tBu protection and Pbf (Arg) and Trt (Asn)

2. C→N construction, FMOC strategy

3. Single coupling (3 eq) with MS analysis at key positions

Strategy 2:

1. Use Convergent solid phase peptide synthesis (CSPPS) and capitalise on repeat section and Gly-Gly cut

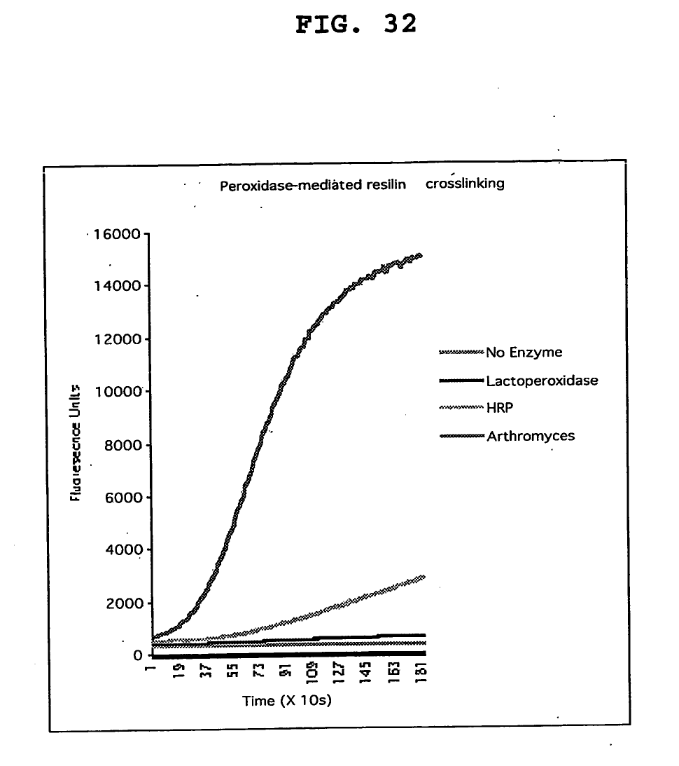

Mass spectrum of complete 26-mer peptide is attached as FIG. 32.

### Example 11

### Cross-Linking of Soluble Resilin Using Peroxidases

Pro-resilin was purified from E. coli cells as described above and was cross-linked into an insoluble polymer in a process which may also be applied to the synthetic resilins of Examples 9 & 10. The formation of the insoluble gel depended on the concentration of the resilin protein solution. For gel formation, soluble resilin monomer was concentrated to 80 mg/ml, 150 mg/ml and 250 mg/ml in 0.25M Borate buffer pH 8.2, as described above.

In order to test the effectiveness of the 3 commercially available peroxidase enzymes, the following small-scale experiment was carried out. Resilin was used at 5 mg/ml. Horseradish peroxidase (Boehringer #814407), Lactoperoxidase (Sigma #L8257) and Arthromyces ramosus peroxidase (Sigma #P4794) were dissolved in buffer at 1 mg/ml. Hydrogen peroxide was prepared from a fresh 30% solution and was used as a (100 mM) stock solution.

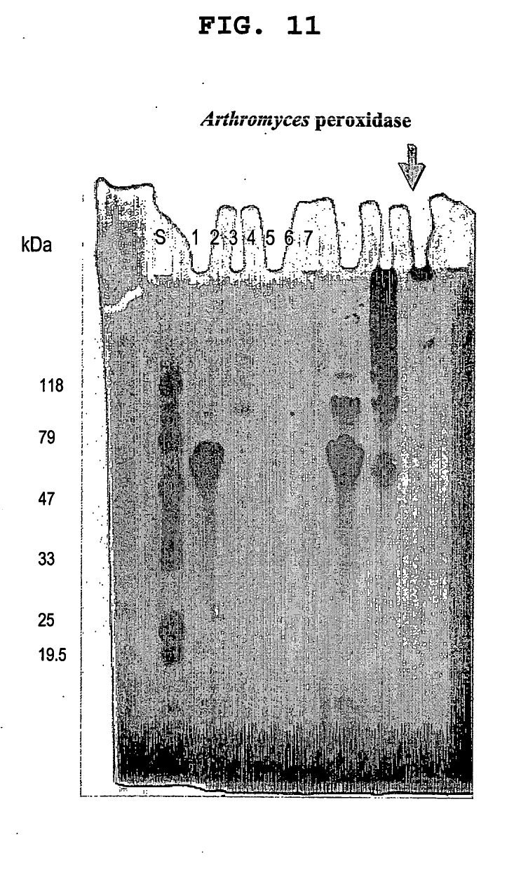

To a solution of purified resilin (20 μl) enzymes were added (2 μl) and the reaction was started by addition of hydrogen peroxide. Final concentrations were therefore: Resilin (5 nmole/40 μl), H₂O₂ (5 mM) and enzymes (40 μmol/40 μl). The reaction was carried out in borate buffer (0.25M) at pH 8.2 at 37° C. for 4 h. Reactions were stopped by the addition of 10 μl of lysis buffer and 10 μl of the mixture was analysed by SDS-PAGE on 10% gels (Invitrogen i-Gel). The results of this experiment are shown in FIG. 11.

Lane 1 shows the purified soluble resilin prior to cross-linking. Lanes 2, 3 and 4 show the peroxidase enzymes used in the experiment while lanes 5, 6 and 7 show, respectively, the effects of lactoperoxidase, horseradish peroxidase and Arthromyces peroxidase on the soluble resilin. Lactoperoxidase was the least effective peroxidase at causing cross-linking of soluble resilin as only a small percentage of the monomer was converted to a dimer. Horseradish peroxidase was more effective as a ladder of higher molecular weight oligomers was apparent by Coomassie blue staining of the gel. In contrast, the Arthromyces peroxidase converted all of the monomer to very large protein polymers which barely entered the 10% polyacrylamide separating gel.

In order to produce insoluble resilin polymer the protein concentration was increased by passage of the soluble resilin through a Centricon™ (10 kDa) filtration device. The protein concentration was increased from 5 mg/ml to 80 mg/ml, 170 mg/ml and 150 mg/ml (8%, 17% and 25% protein solutions, respectively). The reaction conditions were: soluble resilin (40 μl), H₂O₂ (10 mM), peroxidase (5 μl of 10 mg/ml) in 0.25M Borate buffer pH 8.2. reaction was initiated by addition of hydrogen peroxide. An instantaneous gel formation was observed in all three reactions, with the 25% protein solution yielding the firmest gel and the 8% resilin solution gave a very low density gel, which was not completely solid.

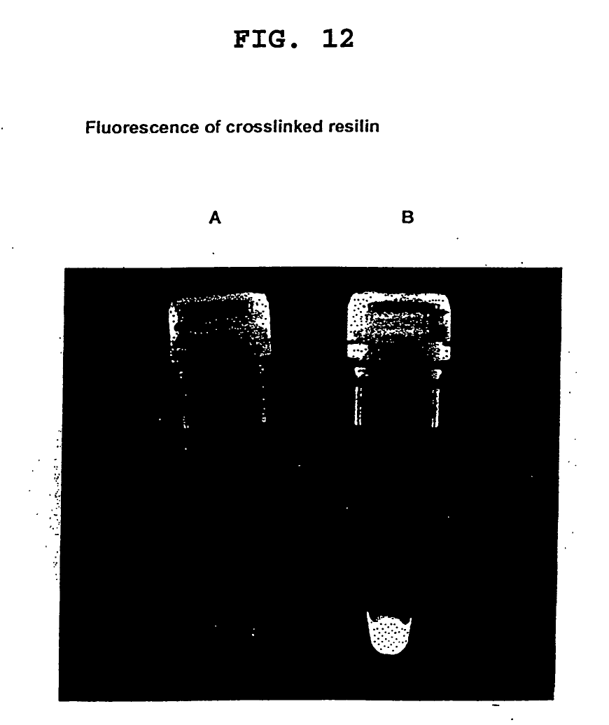

The gel which formed was brightly fluorescent upon irradiation with long-wave (300 nm) UV light (tube B), in comparison with an equivalent quantity of soluble resilin before cross-linking (tube B), as shown in FIG. 12, was insoluble in buffer and water.

These results are consistent with the comparative effectiveness of Arthromyces peroxidase at causing cross-linking of the soluble protein calmodulin into very large polymers (Malencik and Anderson, 1996; Malencik et al, 1996). These authors also showed that the fungal peroxidase was more effective than both horseradish peroxidase and lactoperoxidase.

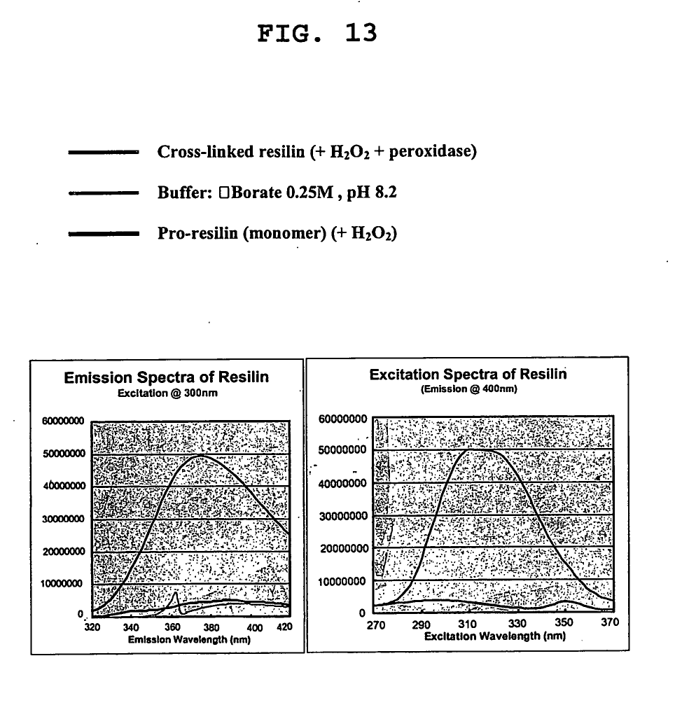

The fluorescence spectrum of the material cross-linked in lane 7, FIG. 4 was obtained in 3 ml of 0.25M borate buffer pH 8.2, using a Perkin-Elmer fluorimeter, with excitation carried out at 300 nm for generation of the emission spectrum and emission monitored at 400 nm for generation of the excitation spectrum. These spectra were compared to those generated an equivalent quantity of uncross-linked soluble resilin. These results are shown in FIG. 13. The spectra show excitation and emission maxima closely resembling the spectra reported for dityrosine standard and for cross-linked calmodulin reported by Malencik and Anderson (1996). These values are: (in borate buffer ph 8.4) Excitation maximum=315 nm; Emission maximum=377 nm. Authentic dityrosine shows maximum sensitivity for excitation at 301 nm and emission at 377 nm in borate buffer. These wavelengths represent the isosbestic and isoemissive points found in the absorption and fluorescence emission spectra of dityrosine in the presence of varying amounts of boric acid-sodium borate buffer (Malencik et al. 1996).

FIG. 32 shows a comparison of dityrosine fluorescence produced by various peroxidases and measured using a microtitre plate fluorescence reader (BMG Polarstar) with excitation at 300 nm and emission at 420 nm. Peroxidases were made up in PBS to 1 mg/ml. Resilin concentration was 5 mg/ml. Peroxide concentration was 5 mM. Reactions were carried out in 100 mM borate buffer pH 8.2. Reactions were carried out at 37 degrees and started by addition of enzyme. These data are supported by the results showing dityrosine formation from L-tyrosine and the cross-linking of calmodulin by Arthromyces ramosus peroxidase (Malencik et al. 1996, Malencik and Anderson).

### Example 12

Purified soluble recombinant resilin was crosslinked by preparing a 20% solution of resilin protein in 100 mM borate buffer pH 8.5 and treating with Arthromyces ramosus peroxidase in the presence of 10 mM H₂O₂ at room temperature. The conditions for rubber formation were:

40 μL resilin solution (200 mg/ml)

5 μL H₂O₂ (100 mM stock solution)

5 μL Arthromyces peroxidase (10 mg/ml)

An instantaneous formation of solid rubber material occurred upon addition of the enzyme.

The soluble protein was converted to a highly fluorescent (excitement λ=320 nm) insoluble material within 5 seconds. This material was washed in 0.1M tris buffer pH 8.0 and tested for comparative resilience using Atomic Force Microscopy (AFM). The samples were dried and then either resuspended in water or maintained at 70% relative humidity for AFM testing. Where humidity control was required this was achieved by enclosing both the sample and the lower portion of the SPM scanner tube with a small Perspex chamber and flushing the system with nitrogen gas of the desired humidity, obtained by bubbling the gas through reverse osmosis water. A Honeywell monolithic integrated humidity sensor and a “K” type thermocouple sensor were inserted through small holes in the end wall of the chamber in order to monitor humidity and temperature. The Butadiene and Butyl rubber were supplied as sheets by Empire Rubber, Australia. The samples had been vulcanised using standard curative systems and contained no fillers.

A Digital Instruments Dimension 3000 Scanning Probe Microscope (SPM) was used to capture Force-Distance curves from which resilience could be determined. Measurements made in air were obtained with the SPM operating in TappingMode™ using silicon “Pointprobes” while Measurements made in water were obtained with the SPM operating in ContactMode™ using “Nanoprobe” Silicon Nitride Probes. Relative triggers of 20-100 nm of deflection were used to limit the cantilever deflection and thus the total force applied to the samples during force-distance measurements. The resilience of the sample was defined as the area under the contact region of the retract curve expressed as a percentage of the area under the contact region of the approach curve. It is inversely related to the hysteresis between the approach and retract portions of the curves. If adhesion occurred between the tip and the sample this was taken into account when measuring the area under the retract curve. Prior to force-distance measurements on the sample, the position-sensitive detector was calibrated by conducting a force-distance measurement on a hard material (glass).

### Example 13

### Gamma Irradiation for Crosslinking Resilin

50 μl aloquots of concentrated resilin (230 mg/ml) was placed into 7 glass tubes. They were exposed to gamma radiation, using a Cobalt-60 source.

Exposure times were for 1, 2, 4, 8, 16, 32 and 64 hours. Exposure was continuous for all samples. (Radiation source=4.5 kG/h).

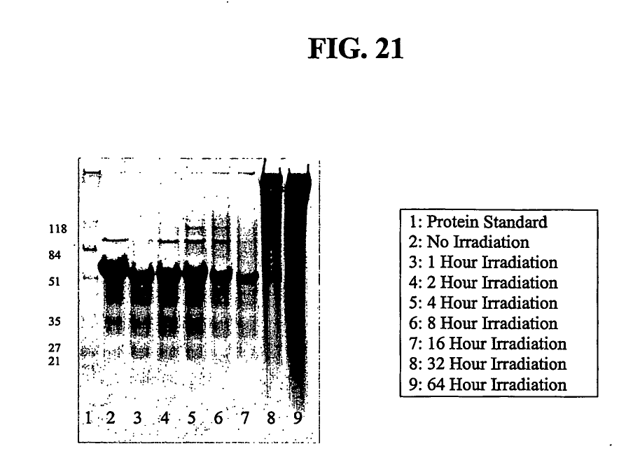

The exposed resilin was diluted 40:1 with 10 mM phosphate buffer pH 8.0. 1 μl of this solution was mixed with 14 μl of loading dye and loaded into each gel well. Note that after 32 and 64 hours of exposure, the resilin could not be pipetted hence a small amount was picked up at the end of a tip and mechanically mixed with the loading dye. A protein standard was used in lane 1. The gel was run at 160V and, once finished, was stained with Coomassie Blue. The resulting gel is shown in FIG. 21.

Resilin monomer runs at around 50 kDa on an SDS-PAGE gel and can be clearly seen as the dominant band in lanes 2-6. Crosslinking between two resilin monomers to create a dimer, will double the size of the protein and hence will run at around 100 kDa. Trimers will run at around 150 kDa and so on. Fully crosslinked resilin should remain at the bottom of the well i.e. the very top of the lane.

The gel shows that crosslinking is taking place after 1 hour irradiation with a faint band at around 100 kDa. However, comparing the relative concentrations of the monomer and dimer shows that not a lot of crosslinking has occurred at this point.

With further exposure, the degree of crosslinking ie the proportion of dimers, increases such that after 16 hours irradiation, the proportion of uncrosslinked resilin is around the same as crosslinked resilin. At 32 and 64 hours, the resilin does not easily progress through the gel indicating that little monomer remains and the resilin contains many crosslinks. A slight band of monomer can be seen in the 32 hour sample.

Therefore, to achieve full crosslinking of resilin using gamma radiation requires at least 32 hours of exposure. This amount of exposure may damage the protein, and therefore this method is not preferred.

### Example 14

### UVB Radiation Crosslinking of Resilin

100 μl of concentrated resilin (230 mg/ml) was diluted in 900 μl PBS (Phosphate Buffer Solution) to give a final concentration of 23 mg/ml. This was aloquoted into 7×100 μl samples in quartz glass cups of 5 mm internal diameter. The cups were sealed with sticky labels, ensuring that this did not hinder the exposure of the resilin solution to the UVB radiation.

The samples were exposed to UVB radiation using UVB tubes designed for a QUV Weatherometer. Samples were located 10 mm from the edge of the UVB tube. This was performed at ambient air temperatures for 1, 2, 4, 8, 16, 32 and 64 hours. All exposures were continuous except for the 16 hour (2×8 hour exposures on consecutive days) and 64 hour (56 hours followed by 8 hours exposure 2 days later) exposures. After exposure, the samples were transferred to eppendorf tubes to minimise loss of water from evaporation.

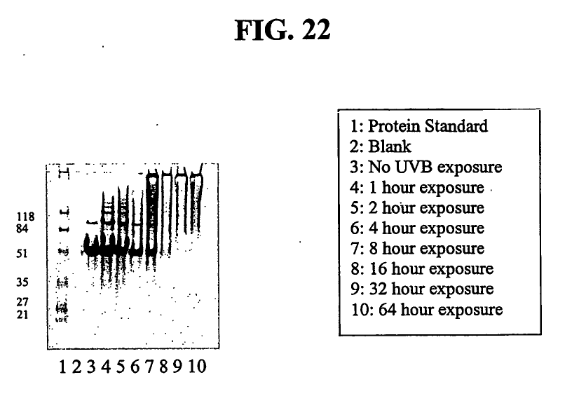

1 μl of each resilin solution was mixed with 14 μl of loading dye, heated to 95° C. for 2 minutes and loaded onto a gel. The results are shown in FIG. 22. The gel shows that a substantial amount of crosslinking takes place within one hour of exposure. There are many dimers, trimers and higher level crosslinks taking place although the volume of monomer is greater than the volume of crosslinked protein. Increasing the exposure to UVB, increases the amount of crosslinking and reduces the volume of monomer. After 8 hours exposure, much of the crosslinked protein remains at the top of the lane suggesting that multiple crosslinks have formed. There is some evidence of dimers however the higher order crosslinks are not evident. This may suggest that a lot of the material is now forming multiple crosslinks. After 16 hours exposure, only a small amount of monomer remains with most of the material remaining at the top of the well hence we have a highly crosslinked protein.

### Example 15

### Riboflavin Crosslinking of Resilin with UVB Radiation

50 μl aloquots of 10 mg/ml resilin in 50 mM TRIS and 50 mM NaCl, were placed into quartz glass cups after 25 μM Riboflavin was added and mixed. The riboflavin was dissolved into distilled water. The samples were exposed to UVB radiation as per previous UVB radiation experiments, for 30, 60, 120 and 240 minutes duration.

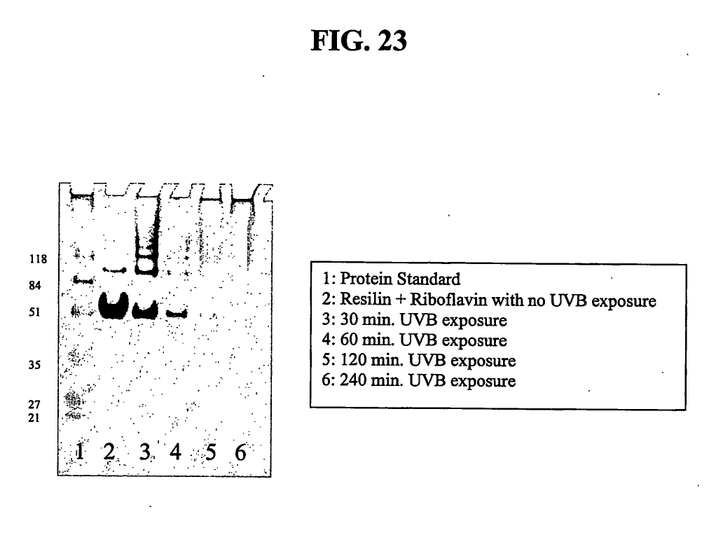

After exposure, 1 μl of each solution was mixed with loading dye, heated to 95° C. and loaded onto an SDS-PAGE gel. The results are shown in FIG. 23. They show that a substantial amount of resilin has crosslinked after just 30 minutes with all resilin monomer being crosslinked after 4 hours exposure. This shows a large improvement in crosslinking time compared with resilin exposed to UVB without riboflavin. A small amount of resilin dimer and trimer exists after 4 hours exposure.

### Example 16

### Fluorescein Crosslinking of Resilin with White Light

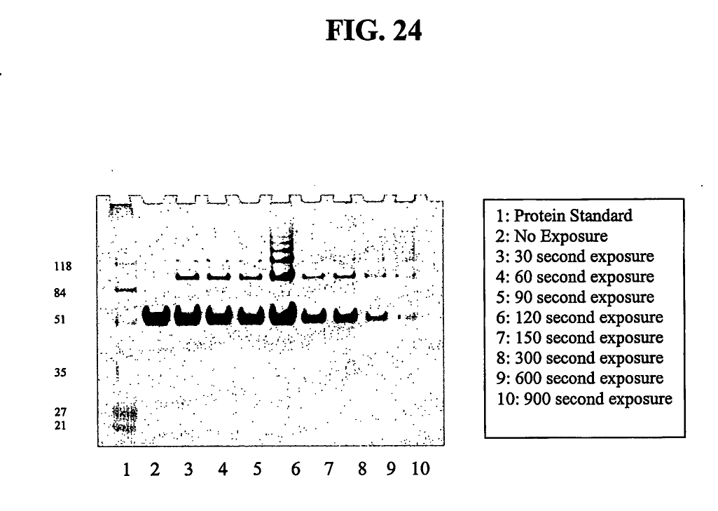

100 mM fluorescein solution was produced using 0.1 mM NaOH in water. 1 μl of the fluorescein solution was mixed with 1 ml of 10 mg/ml resilin. 190 μl of the resilin was aloquoted into 5 wells and kept on ice. The resilin was exposed to 2×300 W globes positioned 10 cm from the top of the wells for 30, 60, 90, 120 and 150 seconds. 1 μl of the resulting solution was mixed with 14 μl of loading dye and run on an. SDS-PAGE gel. The results (not shown here) showed that more time was needed to complete the crosslinking. Therefore, an additional exposures were performed for 150, 300, 600, 900 seconds. With these time-frames, the globes were heating up excessively so the exposures were conducted at 150 second intervals, with 60 second rest intervals to give the globes a chance to cool. The results are shown in FIG. 24. They show that after only 30 seconds, considerable crosslinking has occurred. The reduction in monomer volume decreases considerably after 10 minutes exposure. After 15 minutes exposure, most of the resilin monomer has been crosslinked.

It is expected that after crosslinking, a large proportion of the resilin would remain in the wells however there appears to be little of this material in the wells. The reason for this is due to the aggregation of the crosslinked resilin in solution. The solutions were not prepared for the gel until the following day, allowing the resilin to aggregate. The aggregate could not be pippetted and hence was absent from the loading onto the gel.

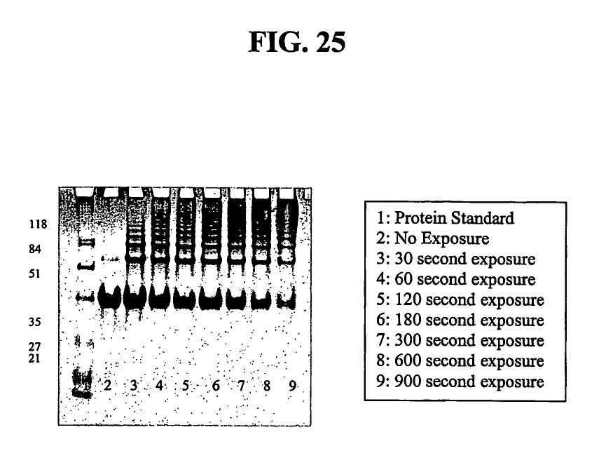

To alleviate this, the experiment was repeated. Exposed resilin was quickly pippetted into the loading dye and a gel run immediately after the final exposure was completed. The results are shown in FIG. 25.

### Example 17

### Coumarin Crosslinking with Ultraviolet Mercury Lamp (380 nm)

Concentrated resilin was diluted to 10 mg/ml with 50 mM TRIS and 50 mM NaCl. The solution was divided into three parts. The first part was mixed with 100 μM 7-hydroxycoumarin-3-carboxylic acid and 90 μl aliquots were placed into small tubes with a black cap. The second part was mixed with 10 μM 7-hydroxycoumarin-3-carboxylic acid and 90 μl aliquots were placed into small tubes with a blue cap. The third part contained no 7-hydroxycoumarin-3-carboxylic acid, and the tubes had red caps.

Each aliquot was exposed to varying times under a Mercury laser (380 nm wavelength) as described in the table below. Each solution was exposed in multiples of 10 second bursts.

| Time | Dosage | | | |
|---|---|---|---|---|
| (sec) | (J/cm²) | Black | Blue | Red |
| 0 | 0 | * | * | |
| 10 | 3.53 | * | * | * |
| 30 | 10.6 | * | * | * |
| 60 | 21.2 | * | * | * |
| 300 | 106 | * | * | * |
| 600 | 212 | * | * | |

* denotes exposure

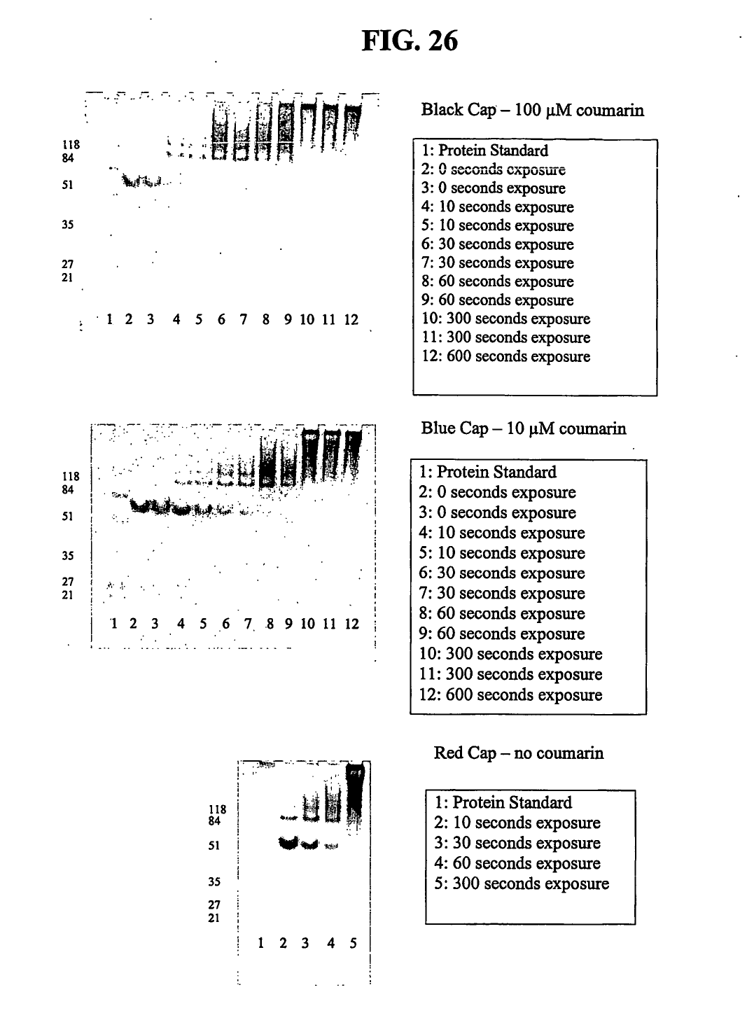

Each exposure condition for the Black and Blue samples was duplicated. After exposure, 5 μl of each solution was mixed with loading buffer and loaded onto SDS-PAGE. The results are shown in FIG. 26.

The results show that all the resilin has been crosslinked after 300 seconds of exposure to the mercury vapour laser. Considerable crosslinking has taken place after 60 seconds. The absence of time intervals between 60 and 300 seconds makes it impossible to determine the exact time required for full crosslinking.

The coumarin appears to have a small effect on the crosslinking. This can be best viewed by comparing the 30 second exposures for resilin containing 100 and 10 μM of coumarin. The sample with a higher concentration of coumarin shows more “smudging” towards the well indicating that more crosslinks have been formed.

### Example 18

### HPLC of Resilin Samples—Dityrosine Analysis

The fluorescein samples from the second experiment described in Example 16 and some samples from solid crosslinked resilin were analysed for dityrosine content via HPLC.

75 μl of each fluorescein sample, and a known weight of crosslinked resilin were digested in 1 ml of 6M HCl containing 0.1% phenol. The samples were heated to 145° C. for 4 hours. Approximately 1 ml of water was added to each sample to make them up to 2 ml. A 400 uL aliquot of each of the samples was evaporated before 400 μl of buffer was added. 20 uL of this volume of solution was injected on the HPLC.

The results are shown in the table below, with samples identified by a time entry in the left-hand column being the samples those described with reference to FIG. 25 in example 16 (the time, in minutes, denoting length of the exposure of the sample to light). Note that the 10 and 15 minute samples may show a lower result than expected due to the agglomeration of the crosslinked resilin at the bottom of the eppendorf tubes. This resulted in an inhomogeneous sample being selected that may have altered the results significantly. Solid B was Resilin crosslinked with enzyme however the crosslinking reaction did not produce a homogenous material. Solid C was Resilin crosslinked with enzyme and produced a more homogenous rubber due to better mixing of the enzyme. Soluble resilin was also tested as a standard with no crosslinking present.

TABLE
Dityrosine analysis in soluble and crosslinked resilin

| | | | | HPLC | | Dityrosine | % |
|---|---|---|---|---|---|---|---|
| Sample | Weight | Volume | HPLC UV | FLU | Dityrosine | FLU | dityrosine |
| name | (mg) | (ml) | (μg/ml) | (μg/ml) | UV (μg/mg) | (μg/mg) | vs tyrosine |
| Solid B | 3.7 | 1.70228 | 9.196 | 10.219 | 4.231 | 4.702 | 15.9 |
| Soluble | 3.5 | 1.69832 | 0 | 0.047 | 0 | 0.023 | 0.57 |
| Solid C | 6.2 | 1.70285 | 27.254 | 30.693 | 7.485 | 8.430 | 18.4 |
| 0 | 1.725 | 1.70818 | 0 | 0.232 | 0 | 0.230 | 0 |
| 0.5 | 1.725 | 1.69987 | 1.294 | 1.411 | 1.275 | 1.390 | 0.84 |
| 1 | 1.725 | 1.6943 | 1.506 | 1.671 | 1.479 | 1.641 | 1.35 |
| 2 | 1.725 | 1.7089 | 2.415 | 2.733 | 2.392 | 2.707 | 1.82 |
| 3 | 1.725 | 1.70468 | 2.401 | 2.664 | 2.373 | 2.633 | 2.01 |
| 5 | 1.725 | 1.71478 | 3.1 | 3.478 | 3.082 | 3.457 | 2.52 |
| 10 | 1.725 | 1.64278 | 2.730 | 2.972 | 2.600 | 2.830 | 4.26 |
| 15 | 1.725 | 1.72742 | 2.669 | 3.02 | 2.673 | 3.024 | 5.21 |

The dityrosine was determine using the following equation: (volume*HPLC/weight) and results in a figure for the amount of dityrosine per weight of protein.

To determine the percentage of tyrosine that had crosslinked to form dityrosine, the area under the tyrosine and dityrosine curves was recorded and the percentage calculated using the following equation: dityrosine area/(dityrosine area+tyrosine area).

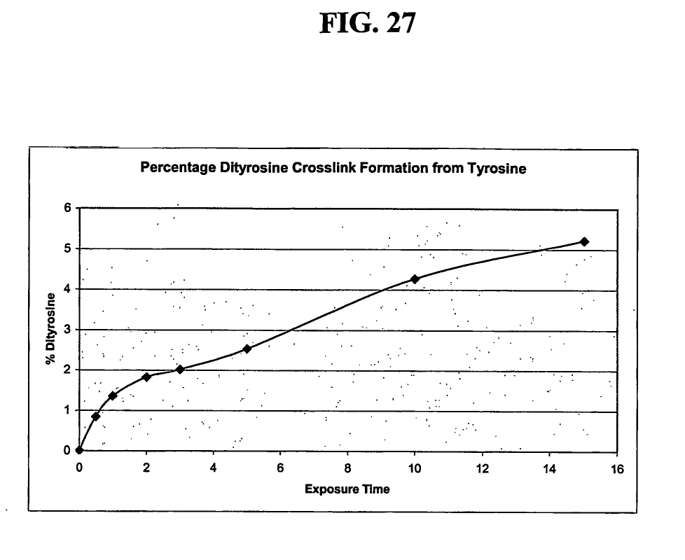

The results (FIG. 27) show that there is a steady increase in the amount of dityrosine with greater exposure to white light when fluorescein is added to the resilin. This indicates that more crosslinks are forming which is in agreement with the results of the SDS PAGE gel.

The amount of tyrosine crosslinking is larger for the enzyme catalysed reaction than for 15 minutes exposure to white light with the addition of fluorescein. In fact, there are at least 3 times as many crosslinks formed.

### Example 19

### Crosslinking of Soluble Resilin Using Tris(2,2′-bipyridyl)Ruthenium(II)dichloride

The PICUP (photo-induced cross-linking of unmodified proteins) reaction is induced by very rapid, visible light photolysis of a tris-bipyridyl Ru(II) complex in the presence of an electron acceptor. Following irradiation, a Ru(III) ion is formed, which serves as an electron abstraction agent to produce a carbon radical within the polypeptide (backbone or side chain), preferentially at positions where stabilization of the radical by hyperconjugation or resonance is favored—tyrosine and tryptophan residues. The radical reacts very rapidly with a susceptible group in its immediate proximity to form a new C—C bond (Fancy and Kodadek, 1999 and Fancy, 2000)

Essentially, the method described by Fancy and Kodadek (1999) was used. This involved preparing a stock solution (0.1M) of Tris(2,2′-bipyridyl)ruthenium dichloride in water. Fresh ammonium persulphate (0.5M) was prepared just prior to use. Recombinant resilin was dialysed in 50 mM Tris/HCl+50 mM sodium phosphate pH 8.0 and concentrated to ca. 200 mg/ml as described in Example 4

The lamp was a 600 W quartz tungsten halogen (2×300 W) (GE #38476 300 W). The spectral output shows a broad peak from 300 nm to 1200 nm.

Oxidative crosslinking of proteins mediated by the tris(2,2′-bipyridyl)ruthenium(II)dichloride ((Ru(II)(bpy)³)²⁺, ammonium persulphate (APS) and visible light was originally described by Fancy and Kodadek (1999). This method preferentially crosslinks associated or self-assembled proteins following brief photolysis. The reaction has been proposed to proceed through a Ru(III) intermediate formed by photoinitiated oxidation of the metal centre by APS. The Ru(III) complex is a potent one-electron oxidant and can oxidise tyrosine (or tryptophan—although there are no trp residues in the resilin—5 sequence) side chains, creating a radical that can couple to appropriate nearby residues by a variety of pathways. One possible crosslinking reaction that can occur is the formation of an arene coupling reaction. If the neighboring amino acid is tyrosine, a dityrosine bond is formed (Fancy and Kodadek, 1999).

Crosslinking of Resilin Exon 1 Soluble Recombinant Protein

Our experiments were carried out in order to investigate the utility of the ((Ru(II)(bpy)³)²⁺, ammonium persulphate (APS) and visible light based method of Fancy and Kodadek (1999). Initially, a 1 mg/ml solution of resilin in PBS (phosphate buffered saline) was used in reactions carried out at room temperature. The APS concentration was 5 mM and the ((Ru(II)(bpy)³)²⁺ concentration was 200 μM. Irradiation was carried out for 10 sec using the 600 W lamp at a distance of 15 cm.

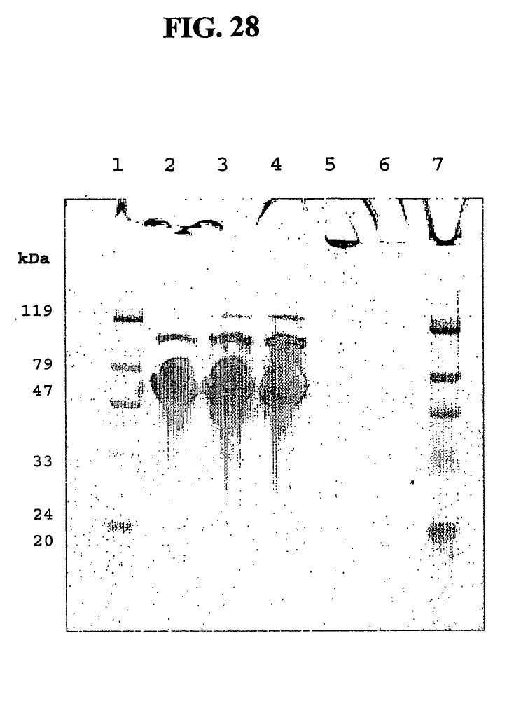

The results of this experiment (FIG. 28) show that there was a quantitative conversion of soluble resilin to a very high molecular weight aggregate which remained at the top of the SDS-PAGE gel. This result suggests that resilin exon 1 recombinant protein is self-associating with tyrosine residues brought into close proximity and available for dityrosine bond formation.

In order to investigate the effect of ((Ru(II)(bpy)³)²⁺ concentration on the crosslinking reaction, an experiment was carried out in which 2-fold serial dilutions of ((Ru(II)(bpy)³)²⁺ were added to a 1 mg/ml solution of soluble resilin in PBS containing 5 mM APS. Irradiation was carried out at room temperature for 10 seconds at a distance of 15 cm.

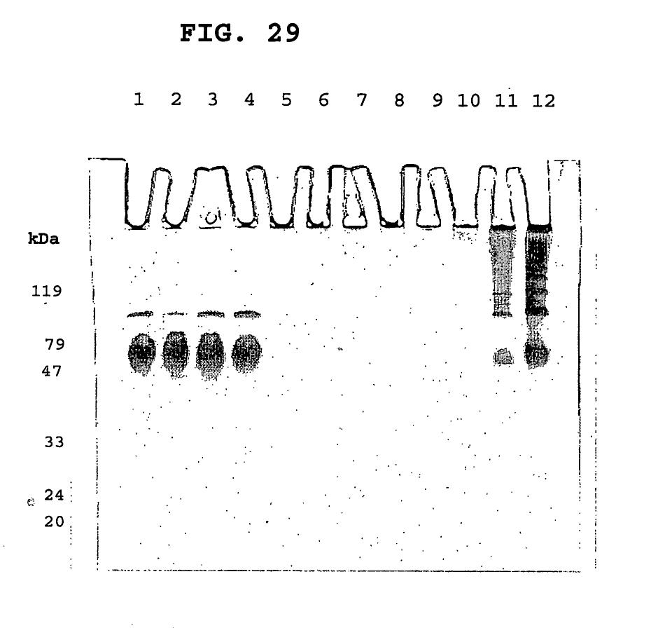

The results showed (FIG. 29) that under these conditions, crosslinking yielded a very high molecular weight product. Furthermore, this experiment revealed the stoichiometry of the reaction in which the Ru(II)Bpy metal salt is oxidized during light illumination. These data show that with 1 mM resilin (40 μM protein) approximately 4 μM Ru(II)Bpy is required to catalyse complete crosslinking.

### REFERENCES

The content of the documents listed hereafter is incorporated herein by reference:
Adams, M. D et al. (2000) The genome sequence of Drosophila melanogaster Science 287 (5461), 2185-2195.
Ali F E, Barnham K J, Barrow C J, Separovic F.
Metal catalyzed oxidation of tyrosine residues by different oxidation systems of copper/hydrogen peroxide. J Inorg Biochem. 2004 January; 98(1):173-84.
Amini F, Denison C, Lin H J, Kuo L, Kodadek T. (2003) Using oxidative crosslinking and proximity labeling to quantitatively characterize protein-protein and protein-Peptide complexes. Chem. Biol. 10: 1115-27.
Andersen, S O and Weis-Fogh, T (1964) Resilin: a rubber-like protein in arthropod cuticle. Adv Insect Physiol 2: 1-65.
Anderson S O (1966) Covalent cross-links in a structural protein, resilin. Acta Physiol Scand Suppl 1966; 263:1-81
Andreev O A, Reshetnyak Y K, Goldfarb R H. (2002) Evidence of inter- and intra-molecular crosslinking of tyrosine residues of calmodulin induced by photo-activation of ruthenium(II). Photochem Photobiol Sci. 2002 October; 1(10):834-6.
Ardell, D H and Andersen, S O (2001) Tentative identification of a resilin gene in Drosophila melanogaster. Insect Biochem Molecular Biol 31: 965-970.
Barro F, Rooke L, Bekes F, Gras P, Tatham A S, Fido R, Lazzeri P A, Shewry P R, Barcelo P. (1997) Transformation of wheat with high molecular weight subunit genes results in improved functional properties. Nat. Biotechnol. 15: 1295-1299.
Bayley, H, Cao, Q and Wang, Y (2000) Molluscan ligament polypeptides and genes encoding them. U.S. Pat. No. 6,127,166.
Bennet-Clark, H C and Lucey, E C A (1967) The jump of the flea. J Exp Biol 47: 59-76.
Coles G C (1966) Studies on resilin biosynthesis. J Insect Physiol June; 12(6):679-691.
Deming, T (1999) Mussel byssus and biomolecular materials. Current opinion in Chemical Biology 3: 100-105.
Edens W A, Sharling L, Cheng G, Shapira R, Kinkade J M, Lee T, Edens H A, Tang X. Sullards C, Flaherty D B, Benian G M, Lambeth J D. (2001) Tyrosine cross-linking of extracellular matrix is catalyzed by Duox, a multidomain oxidase/peroxidase with homology to the phagocyte oxidase subunit gp91phox. J Cell Biol 154: 879-891.
Fancy, D A and Kodadek, T (1999) Chemistry for the analysis of protein-protein interactions: rapid and efficient crosslinking triggered by long wavelength light. Proc Natl Acad Sci (USA) 96: 6020-6024
Fancy D A (2000) Curr Opin Chem. Biol. 4(1):28-33. Elucidation of protein-protein interactions using chemical cross-linking or label transfer techniques.
Fancy D A, Denison C, Kim K, Xie Y, Holdeman T, Amini F, Kodadek T. (2000) Scope, limitations and mechanistic aspects of the photo-induced cross-linking of proteins by water-soluble metal complexes. Chem. Biol. September; 7(9):697-708.
Field J M, Tatham A S, Shewry P R. (1987) The structure of a high-Mr subunit of durum-wheat (Triticum durum) gluten. Biochem J. 247: 215-21.
Gilbert S M, Wellner N, Belton P S, Greenfield J A, Siligardi G, Shewry P R, Tatham A S. (2000) Expression and characterisation of a highly repetitive peptide derived from a wheat seed storage protein. Biochim Biophys Acta. 1479: 135-46.
Gosline, J M (1980) The elastic properties of rubber-like proteins and highly extensible tissues. Symp Soc Exp Biol 36: 331-357.
Hirano H, Obara Y, Katakura K, Ono S. Effects of ultraviolet B irradiation on lenticular riboflavin metabolism and high-molecular-weight-protein aggregation. Ophthalmic Res. 1990; 22(3):183-6.
Kato Y, Kitamoto N, Kawai Y, Osawa T. The hydrogen peroxide/copper ion system, but not other metal-catalyzed oxidation systems, produces protein-bound dityrosine. Free Radic Biol Med. 2001 Sep. 1; 31(5):624-32.
LaFleur, G J, Horiuchi, Y and Wessel, G M (1998) Sea urchin ovoperoxidase: oocyte-specific member of a haem-dependent peroxidase superfamily that functions in the block to polyspermy. Mechanisms of Development 70: 77-89.
Lazaris A, Arcidiacono S, Huang Y, Zhou J F, Duguay F, Chretien N, Welsh E A, Soares J W, Karatzas C N. (2002) Spider silk fibers spun from soluble recombinant silk produced in mammalian cells. Science 295: 472-476.
Lee, Y and Song, K B (2002) Effect of gamma-irradiation on the molecular properties of myoglobin. Journal of Biochemistry and Molecular Biology 35: 590-594.
Malencik, D A and Anderson, S R (1996) Dityrosine formation in calmodulin: Cross-linking polymerisation catalysed by Arthromyces peroxidase. Biochemistry 35: 4375-4386.
Malencik, D A, Sprouse, J F, Swanson, C A and Anderson, S R (1996) Dityrosine: Preparation, isolation and analysis. Analytical Biochemistry 242: 202-213.
Mayo J C, Tan D X, Sainz R M, Lopez-Burillo S, Reiter R (2003) Free Radic Res. 37(5):543-53. Oxidative damage to catalase induced by peroxyl radicals: functional protection by melatonin and other antioxidants. Brown K C, Kodadek T. (2001) Protein cross-linking mediated by metal ion complexes. Met Ions Biol Syst.; 38:351-84
Miles C S, Weigelt J, Stamford N P, Dammerova N, Otting G, Dixon N E (1997) Precise limits of the N-terminal domain of DnaB helicase determined by NMR spectroscopy. Biochem Biophys Res Commun 231: 126-130
Meyer D E and Chilkoti A (2002) Genetically encoded synthesis of protein-based polymers with precisely specified molecular weight and sequence by recursive directional ligation: examples from the elastin-like polypeptide system. Biomacromolecules 3: 357-367.
Nelson R E, Fessler L I, Takagi Y, Blumberg B. Keene D R, Olson P F, Parker C G, Fessler J H. (1994? Peroxidasin: a novel enzyme-matrix protein of Drosophila development. EMBO J. 13: 3438-3447.
Neylon, C, Brown, S E, Kralicek, A V, Miles, C S Love, C A and Dixon, N E (2000) Interaction of the Escherichia coli replication terminator protein (Tus) with DNA: a model derived from DNA-binding studies of mutant proteins bt surface plasmon resonance. Biochemistry 39: 11989-11999.
Ono S, Hirano H. Photosensitized acceleration of riboflavin on the formation of lenticular HMW-protein aggregation. Int J Vitam Nutr Res. 1987; 57(4):401-3.
Parchment, O, Shewry, P R, tatham, A S and Osguthorpe, D J (2001) molecular modeling of unusual spiral structure in elastomeric wheat seed proteins. Cereal chemistry 78: 658-662.
Renugopalakrishnan, V (2002) A 27-mer Tandem Repeat Polypeptide in Bovine Amelogenin: Synthesis and CD Spectra. Journal of Peptide Science J. Peptide Sci. 8: 139-143 (2002).
Rief M, Gautel M, Oesterhelt F, Fernandez J M, Gaub H E. (1997) Reversible unfolding of individual titin immunoglobulin domains by AFM. Science. 276: 1109-1112.
Silva, E and Godoy J (1994) Riboflavin sensitised photooxidation of tyrosine. Int J Vit Nutr Res 64: 253-256.
Spikes, J D, Shen, H R, Kopeckova, P and Kopecek, J (1999) Photodynamic cross-linking of proteins. III. Kinetics of the FMN- and rose bengal-sensitised photooxidation and intermolecular cross-linking of model tyrosine-containing N-(2-hydroxypropyl)methacrylamide copolymers. Photochem Photobiol 70: 130-137.
Stefan U. Egelhaaf, Eric van Swieten, Tjibbe Bosma, Esther de Boef, Alard A. van Dijk, George T. Robillard (2003) Size and shape of the repetitive domain of high molecular weight wheat gluten proteins. I. Small-angle neutron scattering Biopolymers 69: 311-324
Tatham, A S, Drake A F and Shewry, P R (1989) Conformational studies of a synthetic peptide corresponding to the repeat motif of C hordein. Biochem J 259: 471-476.
Tatham, A S and Shewry, P R (2000) Elastomeric proteins: biological roles, structures and mechanisms. Trends Biochem Sci November: 567-571.
Urry, D W (1988) Entropic elastic processes in protein mechanisms. 1. Elastic structures due to an inverse temperature transition and elasticity due to internal chain dynamics. J Prot Chem 7: 1-34.
Urry, D W, Luan, C H and Peng, S Q (1995) Molecular biophysics of elastin. Structure function and pathology. Ciba Found Symp 192: 4-30.
Urry, D W (2001) Bioelastomer nanomachines and biosensors. PCT Application WO 02/00686.
Van Dijk A A, De Boef E, Bekkers A, Van Wijk L L, Van Swieten E, Hamer R J, Robillard G T. (1997) Structure characterization of the central repetitive domain of high molecular weight gluten proteins. II. Characterization in solution and in the dry state. Protein Sci. March; 6(3):649-56.
Vollrath F, Knight D P. (2001) Liquid crystalline spinning of spider silk. Nature 410: 541-548.
Wang, C., Stewart R J. and KopecÏek J (1999) Hybrid hydrogels assembled from synthetic polymers and coiled-coil protein domains. Nature 397: 417-420.
Webster A, Britton D, Apap-Bologna A, Kemp G. (1989) A dye-photosensitized reaction that generates stable protein-protein crosslinks. Anal Biochem 179(1):154-7.
Weis-Fogh, T (1960) A rubber-like protein in insect cuticle. J Exp Biol 37: 889-907.
Weis-Fogh, T (1961a) Thermodynamic properties of resilin, a rubber-like protein. J Mol Biol 3: 520-531.
Weis-Fogh, T (1961b) Molecular interpretation of the elasticity of resilin, a rubber-like protein. J Mol Biol 3: 648-667.
Woody, R W (1986) Woody R W. In Peptide, Polypeptides, and Proteins, John Wiley and Sons: New York, 1974; 338-358.
Zhao X, Smartt C T, Li J, Christensen B M.9. (2001) Aedes aegypti peroxidase gene characterization and developmental expression. Insect Biochem Mol Biol 31: 481-90.

1. A bioelastomer which is a polypeptide comprising a plurality of repeat units with the consensus sequence SXXYGXP, where S is serine, X is an amino acid, Y is tyrosine, G is glycine and P is proline, and which is cross-linked through dityrosine bond formation, with the proviso that the bioelastomer is not resilin.

2. A bioelastomer as claimed in claim 1 which comprises a plurality of repeat units with the consensus sequence:
X₁X₂X₃X₄SX₅YGX₇PX₈X₉X₁₀X₁₁
wherein:
X₁ is absent or any amino acid;
X₂ is absent or any amino acid;
X₃ is absent or any amino acid;
X₄ is P or S;
X₅ is a charged or polar amino acid;
X₆ is a charged or polar amino acid;
X₇ is A or P;
X₈ is G or A;
X₉ is absent, G or a polar amino acid;
X₁₀ is absent, G or a polar amino acid; and
X₁₁ is absent or any amino acid.

3. A bioelastomer as claimed in claim 2 wherein X₁, if present, is G, Y, A or N.

4. A bioelastomer as claimed in claim 2 wherein X₂, if present, is a basic amino acid or G.

5. A bioelastomer as claimed in claim 2 wherein X₃, if present, is a basic amino acid, T or P.

6. A bioelastomer as claimed in claim 2 wherein X₄ is P.

7. A bioelastomer as claimed in claim 2 wherein X₅ is D, T or S.

8. A bioelastomer as claimed in claim 2 wherein X₆ is S, Q or T.

9. A bioelastomer as claimed in claim 2 wherein X₇ is A.

10. A bioelastomer as claimed in claim 2 wherein X₈ is G.

11. A bioelastomer as claimed in claim 2 wherein X₉, if present, is G, Q or S.

12. A bioelastomer as claimed in claim 2 wherein X₁₀, if present, is G, S or N.

13. A bioelastomer as claimed in claim 2 wherein X₁₁, if present, is G, Q, P, S or N.

14. A bioelastomer as claimed in claim 2 wherein at least one of X₈, X₉ and X₁₀ is G.

15. A bioelastomer as claimed in claim 2 comprising repeat units having the amino acid sequence GGRPSDSYGAPGGGN or GGRPSDTYGAPGGGN.

16. A bioelastomer as claimed in claim 2 comprising repeat units of the amino acid sequence PSSQYGAPAQT.

17. A bioelastomer as claimed in claim 2 comprising repeat units including the amino acid sequence SSSYGAP or SSTYGAP or STTYGAP.

18. A bioelastomer as claimed in claim 1 in which dityrosine is formed by enzyme-mediated cross-linking.

19. A bioelastomer as claimed in claim 18 wherein the enzyme is a peroxidase.

20. A bioelastomer as claimed in claim 19 wherein the peroxidase is Arthromyces peroxidase.

21. A bioelastomer as claimed in claim 1 wherein dityrosine is formed by photo-induced cross-linking by photolysis of a tris-bipyridyl Ru(II) complex in the presence of an electron acceptor.

22. A bioelastomer as claimed in claim 1 wherein dityrosine is formed by irradiation with gamma radiation, UVB or visible light.

23. A bioelastomer as claimed in claim 1 comprising one cross-link for every 5 to 100 amino acids.

24. A bioelastomer as claimed in claim 23 comprising one cross-link for every 20 to 50 monomer units.

25. An isolated polypeptide comprising a plurality of repeat units having the consensus sequence SXXYGXP, where S is serine, X is an amino acid, Y is tyrosine, G is glycine and P is proline, with the proviso that the polypeptide is not pro-resilin.

26. An isolated polypeptide as claimed in claim 25 comprising a plurality of repeat units as set forth in claim 2.

27. A polypeptide as claimed in claim 25 comprising the amino acid sequence set forth in SEQ ID NO:25, 27, 29 or 31.

28. An isolated nucleic acid encoding a polypeptide as claimed in claim 25.

29. An isolated nucleic acid having the nucleotide sequence set forth in any one of SEQ ID NO:17-24, 26, 28 or 30.

30. A method of preparing a bioelastomer as claimed in claim 1, comprising the steps of:
(1) providing a polypeptide as claimed in claim 25;
(2) initiating a cross-linking reaction; and
(3) isolating the bioelastomer

31. A hybrid molecule comprising a polypeptide as claimed in claim 25 and a second polymeric molecule, preferably selected from the group consisting of mussel byssus protein, Spider silk protein, collagen, elastin, and fibronectin, or fragments thereof.

32. A nanomachine comprising a polypeptide as claimed in claim 25 acting as a spring mechanism and a device upon which said spring mechanism acts.

33. A biosensor comprising a polypeptide as claimed in claim 25 or a bioelastomer as claimed in claim 1 or a hybrid molecule as claimed in claim 31.

34. A manufactured article consisting of or comprising a bioelastomer as claimed in claim 1 or a hybrid molecule as claimed in claim 31.


SEQUENCE LISTING (32 sequences)

SEQ ID NO: 1 - protein, 620 aa, Drosophila melanogaster
MFKLLGLTLLMAMVVLGRPEPPVNSYLPPSDSYGAPGQSGPGGRPSDSYGAPGGGNGGRPSDSYGAPGQGQGQGQGQGGYAGKPSDTYGAPGGGNGNGGRPSSSYGAPGGGNGGRPSDTYGAPGGGNGGRPSDTYGAPGGGGNGNGGRPSSSYGAPGQGQGNGNGGRSSSSYGAPGGGNGGRPSDTYGAPGGGNGGRPSDTYGAPGGGNNGGRPSSSYGAPGGGNGGRPSDTYGAPGGGNGNGSGGRPSSSYGAPGQGQGGFGGRPSDSYGAPGQNQKPSDSYGAPGSGNGNGGRPSSSYGAPGSGPGGRPSDSYGPPASGSGAGGAGGSGPGGADYDNDEPAKYEFNYQVEDAPSGLSFGHSEMRDGDFTTGQYNVLLPDGRKQIVEYEADQQGYRPQIRYEGDANDGSGPSGPGGPGGQNLGADGYSSGRPGNGNGNGNGGYSGGRPGGQDLGPSGYSGGRPGGQDLGAGGYSNGKPGGQDLGPGGYSGGRPGGQDLGRDGYSGGRPGGQDLGASGYSNGRPGGNGNGGSDGGRVIIGGRVIGGQDGGDQGYSGGRPGGQDLGRDGYSSGRPGGRPGGNGQDSQDGQGYSSGRPGQGGRNGFGPGGQNGDNDGSGYRY

SEQ ID NO: 2 - DNA, 2670 bp, Drosophila melanogaster
gtcatagcaaccactgaaccactggaacactgaaccactcgcttgggagagtgagccgcggcagacgccgccgacacattcacccagtggagtagaccgagtagactactttgggccaacgcaacggcggatgcaaaaaggtttggggcccgggcacaatcaccacctcgccgcgcctcgagaagatgtgcaccgaaagaaggcaaatcagcccgagacccaagaccgaggcaccctttacgtggcaatctggcgcagatcggggtggtgaagtgttggcaagcaagctcagtgattgagatttttgctttcgaccgcatagaatcagccaccgcttctctgaagacgatcgtatgtggcagcctcatcagctgcatcagcagcaacagcagcagcagcagctgttgcgctggttgccacacgctccacaaaaccgccacctccaactcatagtcgagttcgtgtgtgtgcatcctagactttcagtttttcggttttgcctcatataaatacgccatgtggcagctaccacagatcagtcagaattcggcgctgcttccaagcgctagtctcatcccaaagatttcgatcagtccgtagaccgagcttcagttccagcttcagctttagatacaagtggaatatgttcaagttactcggcttgacgctgctcatggcaatggtggtccttgggcgaccggagccaccagttaactcgtatctacctccgtccgatagctatggagcaccgggtcagagtggtcccggcggcaggccgtcggattcctatggagctcctggtggtggaaacggtggacggccctcagacagctatggcgctccaggccagggtcaaggacagggacaaggacaaggtggatatgcaggcaagccctcagatacctatggagctcctggtggtggaaatggcaacggaggtcgtccatcgagcagctatggcgctcctggcggtggaaacggtggtcgtccttcggatacctacggtgctcctggtggcggaaatggtggacgcccatcggacacttatggtgctcctggtggtggtggaaatggcaacggcggacgaccttcaagcagctatggagctcctggtcaaggacaaggcaacggaaatggcggtcgctcatcgagcagctatggtgctcctggcggtggaaacggcggtcgtccttcggatacctacggtgctcccggtggtggaaacggtggtcgtccttcggatacttacggcgctcctggtggcggcaataatggcggtcgtccctcaagcagctacggcgctcctggtggtggaaacggtggtcgtccatctgacacctatggcgctcctggtggcggtaacggaaacggcagcggtggtcgtccttcaagcagctatggagctcctggtcagggccaaggtggatttggtggtcgtccatcggactcctatggtgctcctggtcagaaccaaaaaccatcagattcatatggcgcccctggtagcggcaatggcaacggcggacgtccttcgagcagctatggagctccaggctcaggacctggtggccgaccctccgactcctacggacccccagcttctggatcgggagcaggtggcgctggaggcagtggacccggcggcgctgactacgataacgatattgtggagtatgaagccgaccagcagggctaccggccacagatccgctacgaaggcgatgccaacgatggcagtggtcccagcggtcctggaggtcctggcggtcagaatcttggtgccgatggctactccagtggacgtcccggcaatggaaatggcaacggaaatggcggttactccggtggacgtccaggaggccaggatttgggacctagtggatattccggtggtcgtccaggaggtcaggatctaggcgccggtggctactccaatggcaagccgggcggccaagacttgggaccaggcggttactccggtggtcgccctggaggtcaggacttgggtcgagacggctactccggtggacgtccaggtggacaggacctcggtgccagcggctactccaatggtaggccaggcggcaatggcaacggtggatccgatggcggtcgtgtgatcatcggtggacgtgtgataggcggccaggatggcggtgatcagggctactccggcggacgtcccggtggtcaggatcttggacgtgatggctactccagcggtcgtcctggtggtcggccaggcggcaacggccaggatagtcaggatggccaaggatactcgagcggcaggccgggtcagggtggccggaatggattcggacccggtggtcagaacggtgacaacgatggcagcggttatcggtactaggaacgactcacagacacatttagctagattccccgaacatatagcaatacctagtgttaagtccttctagaactatgtcccctacctatgcccctcatccttccgcatctattcgtgcccattctatcccacttcttgctgtctttcgcccatccattcagcctgctcaattcctcgtagtcgtaagtccctgttgatctaaatgcaagtgcgaaaaataacccaaaaaaaggaatgaaatgataaagctacttccgatgtaagtaaagaaattaaatacatatatattttttataaaa

SEQ ID NO: 3 - protein, 310 aa, Drosophila melanogaster
MHHHHHHPEPPVNSYLPPSDSYGAPGQSGPGGRPSDSYGAPGGGNGGRPSDSYGAPGQGQGQGQGQGGYAGKPSDTYGAPGGGNGNGGRPSSSYGAPGGGNGGRPSDTYGAPGGGNGGRPSDTYGAPGGGGNGNGGRPSSSYGAPGQGQGNGNGGRSSSSYGAPGGGNGGRPSDTYGAPGGGNGGRPSDTYGAPGGGNNGGRPSSSYGAPGGGNGGRPSDTYGAPGGGNGNGSGGRPSSSYGAPGQGQGGFGGRPSDSYGAPGQNQKPSDSYGAPGSGNGNGGRPSSSYGAPGSGPGGRPSDSYGPPASG

SEQ ID NO: 4 - protein, 585 aa, Anopheles gambiae
MVAFRRTHLTALVVVCCAVLVPVRTASTSLAKREAPLPPSGSYLPPSGGAGGYPAAQTPSSSYGAPTGGAGSWGGNGGNGGRGHSNGGGSSFGGSAPSAPSQSYGAPSFGGQSSGGFGGHSSGGFGGHSSGGHGGNGNGNGNGYSSGRPSSHYGVPAAPSQSYGAPAQQHSNGGNGGYSSGRPSTQYGAPAQSNGNGFGNGRPSSSYGAPARPSTQYGAPSAGNGNGYAGNGNGRSYSNGNGNGHGNGHSNGNGNNGYSRGPARQPSQQYGPPAQAPSSQYGAPAQTPSSQYGAPAQTPSSQYGAPAQTPSSQYGAPAQTPSSQYGAPAPSRPSQQYGAPAPSRPSQQYGAPAQTPSSQYGAPAQTPSSQYGAPAQTPSSQYGAPAQTPSSQYGAPAQQPSSQYGAPAPSRPSQQYGAPAQQPSAQYGAPAQTPSSQYGAPAPSRPSQQYGAPAQAPSSQYGAPAPSSQYGAPAQQPSSQYGAPAQTPSSQYGAPSFGPTGGASFSSGNGNVGGSYQVSSTGNGFSQASFSASSFSPNGRTSLSAGGFSSGAPSAQSAGGYSSGGPSQVPATLPQSYSSNGGYNY

SEQ ID NO: 5 - protein, 238 aa, Apis mellifera
MKEKTREKYSFLKFRIEIIYFSNQIKTHSSFKYINLTKRLFTAPISGSYLPPSTSYGTPNLGGGGPSSTYGAPSGGGGGRPSSSYGAPSSTYGAPSSTYGAPSNGGGRPSSTYGAPSNGGGRPSSSYGAPSSSYGAPSSTYGAPSNGGGRPSSSYGAPSFGGGGGFGGGNGLSTSYGAPSRGGGGGGGSISSSYGAPTGGGGGGPSTTYGAPNGGGNGYSRPSSTYGTPSTGGGSFGG

SEQ ID NO: 6 - protein, 314 aa, Apis mellifera
SREISKFRSSHPFKFRSFRLQIGLAVFVLALTLVRSEPPVNSYLPPSGNGNGGGGGGSSNVYGPPGFDGQNGIGEGDNGRNGISNSYGVPTGGNGYNGDSSGNGRPGTNGGRNGNGNGRGNGYGGGQPSNSYGPPSNGHGGNGAGRPSSSYGAPGGGNGFAGGSNGKNGFGGGPSSSYGPPENGNGFNGGNGGPSGLYGPPGRNGGNGGNGGNGGRPSGSYGTPERNGGRLGGLYGAPGRNGNNGGNGYPSGGLNGGNGGYPSGGPGNGGANGGYPSGGSNGDNGGYPSGGPNGNGNGNGGYGQDENNVSPYSQ

SEQ ID NO: 7 - protein, 914 aa, Drosophila melanogaster
MRSFGREVAHAPWWTLLPLWLLVVSVVALSLGCLVAPTKSSSVVRIRPSAEHRLQSAPKNLKEAAGAHENVRLICIRSSSTSSSSPGEAASVMRVQLFGVIAVAVVAVSLVRDVGAEPPVNNAYLPPSSPQRPSSKYGAPPVSSYLPPASGPAPSFNSAPSSSYAAPSQSASSGGPYPAAAPRPSSSYGPPASRPSSSYGPPPSRPSQSYGPPPQAKKPHHRRPSSSYGAPRPAPPSQSYGAPPSASYGPPKSAPPSQSYGAPAPPSSKYGPPKSAPSSSYGAPRPAAPSSSYGAPAPPSSSYGAPAAPSSSYGAPAAPSSSYGAPAAPSSSYGAPAPPSKSYGAPAPPSSSYGAPAAPSKSYGAPAPPSSSYGAPAPPSSSYGAPAPPSPSYGAPAPPSKSYGAPAPPSSSYGAPAAPSKSYGAPAPPSSSYGAPAPPSSSYGAPSAPSSSYGPPKPAPAPPSSSYGAPPQAPVSSYLPPASRPSKPSSSYGAPSVSSFVPLPSAPSTNYGAPSKTQSLGSSGYSSGPSSSYEAPVAPPSSSYGAPSSSFQPISPPSSSYGAPSSGSGSSSGSFSAAPSSLYSAPSKGSSGGSFQSAPSSSYSAPSASANSGGSYPSAPSSSYSAPSSSSSSGGPYASAPSSSYSAPSSGSNSGGPYPAAPSSSYSAPSASANSGGSYPSAPSSSYSAPSPGSNSGGPYPAAPSSSYSAPSPSANSGGPYASAPSSSYSAPSSSSNSGGPYAAAPSSSYSAPSSSSSSGGPYPSAPSSSYSAPSSSLSSGGPYPSAPSSSYAAPSPSSNSGGPYPAAPSNSYSAPIAPPSSSYGAPASGPSPSFSAPSSSYGAPSTGSGSSSFSSSSSSFSGASSSSSAGYPSAPSSSYGAPSTGSGHSFSSAPSSSYSAPPAGGSSSSGPYP

SEQ ID NO: 8 - DNA, 265 bp, Ctenocephalides felis
aaacggcggtgctattggacagcctagctcatcttacggagctccaggccagaatggtaacggtggcggcttaagttcaacatatggagcaccaggagcaggcaatggtggcttcggcggtaacggcggtggtctcagctctacatatggtgcaccaggatcaggcaacggtggatttggaggtaacggacttagctctacatatggcgctcctggatccggaaatggaggcttcggtggtaatggaggtgggctgagctcaaca

SEQ ID NO: 9 - protein, 88 aa, Ctenocephalides felis
NGGAIGQPSSSYGAPGQNGNGGGLSSTYGAPGAGNGGFGGNGGGLSSTYGAPGSGNGGFGGNGLSSTYGAPGSGNGGFGGNGGGLSST

SEQ ID NO: 10 - DNA, 593 bp, Haematobia irritans exigua
tggttccggaggttctaatggtaatggtggtggtagaccatcttcttcttatggtgctcctggtgctggcggttccaatggtaatggcggtggtagaccatcttcttcttatggtgctcccggcgctggtggttccaatggtaatggtggtggtagaccttcttcgtcatatggggctcctggagcaggcggttctaatggcaatggtggcggtagaccttcctcgtcatatggtgctcctggtgctggaggttccaatggtaatggcggaagtcgaccttcttccacatatggggcccctggagcgggtggttctaatggtaatggtggtggtaataaaccatcatctagctatggagctccaagtgctggttctaatggtaatggtggatccgagcaaggaagtagcggcagtccatcggatagttatggaccacctgcttcaggtacaggccgtggacgcaatggtggaggtggtggtgccggtggtggtcgaggcggacaacccaatcaagagtatctacctcccaatcaaggtgataatggaaacaatggtggctctggaggtgatgatggttatgattattcccaaa

SEQ ID NO: 11 - protein, 197 aa, Haematobia irritans exigua
GSGGSNGNGGGRPSSSYGAPGAGGSNGNGGGRPSSSYGAPGAGGSNGNGGGRPSSSYGAPGAGGSNGNGGGRPSSSYGAPGAGGSNGNGGSRPSSTYGAPGAGGSNGNGGGNKPSSSYGAPSAGSNGNGGSEQGSSGSPSDSYGPPASGTGRGRNGGGGGAGGGRGGQPNQEYLPPNQGDNGNNGGSGGDDGYDYSQ

SEQ ID NO: 12 - protein, 63 aa, Ctenocephalides felis
YGAPGAGNGGFGGNGGGLSSTYGAPGSGNGGFGGNGLSSTYGAPGSGNGGFGGNGGGLSLTYG

SEQ ID NO: 13 - protein, 62 aa, Haematobia irritans exigua
YGAPGAGGSNGNGGGRPSSSYGAPGAGGSNGNGGGRPSSSYGAPGAGGSNGNGGSRPSSTYG

SEQ ID NO: 14 - protein, 61 aa, Drosophila melanogaster
YGAPGGGNGGRPSDTYGAPGGGNGGRPSDTYGAPGGGGNGNGGRPSSSYGAPGQGQGNGNG

SEQ ID NO: 15 - protein, 64 aa, Anopheles gambiae
YGAPAQTPSSQYGAPAQTPSSQYGAPAQTPSSQYGAPAQQPSSQYGAPAPSRPSQQYGAPAQQP

SEQ ID NO: 16 - protein, 64 aa, Apis mellifera; identical to SEQ ID NO: 15

SEQ ID NO: 17 - DNA, 329 bp, Synthetic
aaccccgtctagccagtatggtgcaccggcgcaaaccccgtctagccagtatggtgcaccggcgcaaaccccgtctagccagtatggtgcaccggcgcaaaccccgtctagccagtatggtgcaccggcgcaaaccccgtctagccagtatggtgcaccggcgcaaaccccgtctagccagtatggtgcaccggcgcaaaccccgtctagccagtatggtgcaccggcgcaaaccccgtctagccagtatggtgcaccggcgcaaaccccgtctagccagtatggtgcaccggcgcaaaccccgtctagccagtatggtgcaccggcgc

SEQ ID NO: 18 - DNA, 268 bp, Synthetic
gcgccaaaccccgtctagccagtatggtgcaccggcgcaaaccccgtctagccagtatggtgcaccggcgcaaaccccgtctagccagtatggtgcaccggcgcaaaccccgtctagccagtatggtgcaccggcgcaaaccccgtctagccagtatggtgcaccggcgcaaaccccgtctagccagtatggtgcaccggcgcaaaccccgtctagccagtatggtgcaccggcgcaaaacccgtctagccagtatggtgcaccggcg

SEQ ID NO: 19 - DNA, 398 bp, Synthetic
gcaaaccccgtctagccagtatggtgcaccggcgcaaaccccgtctagccagtatggtgcaccggcgcaaaccccgtctagccagtatggtgcaccggcgcaaaccccgtctagccagtatggtgcaccggcgcaaaccccgtctagccagtatggtgcaccggcgcaaaccccgtctagccagtatggtgcaccggcgcaaaccccgtctagccagtatggtgcaccggcgcaaaccccgtctagccagtatggtgcaccggcgcaaaccccgtctagccagtatggtgcaccggcgcaaaccccgtctagccagtatggtgcaccggcgcaaaccccgtctagccagtatggtgcaccggcgcaaaccccgtctagccagtatggtgcaccggcgc

SEQ ID NO: 20 - DNA, 328 bp, Synthetic
aaccccgtctagccagtatggtgcaccggcgcaaaccccgtctagccagtatggtgcaccggcgcaaaccccgtctagccagtatggtgcaccggcgcaaaccccgtctagccagtatggtgcaccggcgcaaaccccgtctagccagtatggtgcaccggcgcaaaccccgtctagccagtatggtgcaccggcgcaaaccccgtctagccagtatggtgcaccggcgcaaaccccgtctagccagtatggtgcaccggcgcaaaccccgtctagccagtatggtgcaccggcgcaaaccccgtctagccagtatggtgcaccggcg

SEQ ID NO: 21 - DNA, 433 bp, Synthetic
gcgcaaaccccgtctagccagtatggtgcaccggcgcaaaccccgtctagccagtatggtgcaccggcgcaaaccccgtctagccagtatggtgcaccggcgcaaaccccgtctagccagtatggtgcaccggcgcaaaccccgtctagccagtatggtgcaccggcgcaaaccccgtctagccagtatggtgcaccggcgcaaaccccgtctagccagtatggtgcaccggcgcaaaccccgtctagccagtatggtgcaccggcgcaaaccccgtctagccagtatggtgcaccggcgcaaaccccgtctagccagtatggtgcaccggcgcaaaccccgtctagccagtatggtgcaccggcgcaaaccccgtctagccagtatggtgcaccggcgcaaaccccgtctagccagtatggtgcaccggcgc

SEQ ID NO: 22 - DNA, 235 bp, Synthetic
gcgcaaaccccgtctagccagtatggtgcaccggcgcaaaccccgtctagccagtatggtgcaccggcgcaaaccccgtctagccagtatggtgcaccggcgcaaaccccgtctagccagtatggtgcaccggcgcaaaccccgtctagccagtatggtgcaccggcgcaaaccccgtctagccagtatggtgcaccggcgcaaaccccgtctagccagtatggtgcaccggcgc

SEQ ID NO: 23 - DNA, 499 bp, Synthetic
gcgcaaaccccgtctagccagtatggtgcaccggcgcaaaccccgtctagccagtatggtgcaccggcgcaaaccccgtctagccagtatggtgcaccggcgcaaaccccgtctagccagtatggtgcaccggcgcaaaccccgtctagccagtatggtgcaccggcgcaaaccccgtctagccagtatggtgcaccggcgcaaaccccgtctagccagtatggtgcaccggcgcaaaccccgtctagccagtatggtgcaccggcgcaaaaccccgtctagccagtatggtgcaccggcgcaaaccccgtctagcagtatggtgcaccggcgcaaaccccgtctagccagtatggtgcaccggcgcaaaccccgtctagccagtatggtgcaccggcgcaaaccccgtctagccagtatggtgcaccggcgcaaaccccgtctagccagtatggtgcaccggcgcaaaccccstcwagccagwatggtgcaccggcgc

SEQ ID NO: 24 - DNA, 623 bp, Synthetic
caaaccccgtctagccagtatggtgcaccggcgcaaaccccgtctagccagtatggtgcaccggcgcaaaccccgtctagccagtatggtgcaccggcgcaaaccccgtctagccmgtatggtgcaccggcgcaaaccccgtctagccagtatggtgcaccggcgcaaaccccgtctagccagtatggtgcaccggcgcaaaccccgtctagccagtatggtgcaccggcgcaaaccccgtctagccagtatggtgcaccggcgcaaaccccgtctagccagtatggtgcaccggcgcaaaccccgtctagccagtatggtgcaccggcgcaaaccccgtctagccagtatggtgcaccggcgcaaaccccgtctagccagtatggtgcaccggcgcaaaccccgtctagccagtatggtgcaccggcgcaaaccccgtctagccagtatggtgcaccggcgcaaaccccgtctagccagtatggtgcaccggcgcaaaccccgtctagccagtatggtgcaccggcgcaaaccccgtctagccagtatggtgcaccggcgyaaaccccgtctagccagtatggtgcaccggcgcaaaccccgtctagcyagtatggtgcacc

SEQ ID NO: 25 - protein, 207 aa, Synthetic
QTPSSQYGAPAQTPSSQYGAPAQTPSSQYGAPAQTPSSQYGAPAQTPSSQYGAPAQTPSSQYGAPAQTPSSQYGAPAQTPSSQYGAPAQTPSSQYGAPAQTPSSQYGAPAQTPSSQYGAPAQTPSSQYGAPAQTPSSQYGAPAQTPSSQYGAPAQTPSSQYGAPAQTPSSQYGAPAQTPSSQYGAPAQTPSSQYGAPAQTPSSQYGA

SEQ ID NO: 26 - DNA, 178 bp, Synthetic
gtccgagcgatacctatggtgcgccgggtggtggcaacggtggtcgtccgagcgatacctatggtgcgccgggtggtggcaacggtggtcgtccgagcgatacctatggtgcgccgggtggtggcaacggtggtcgtccgagcgatacctatggtgcgccgggtggtggcaacggtgg

SEQ ID NO: 27 - protein, 59 aa, Synthetic
RPSDTYGAPGGGNGGRPSDTYGAPGGGNGGRPSDTYGAPGGGNGGRPSDTYGAPGGGNG

SEQ ID NO: 28 - DNA, 316 bp, Synthetic
gccatatgcatcaccatcaccatcacgccagtgtgctggtattcagggcgcaaaccccgtctagccagtatggtgcaccggcgcaaaccccgtctagccagtatggtgcaccggcgcaaaccccgtctagccagtatggtgcaccggcgcaaaccccgtctagccagtatggtgcaccggcgcaaaccccgtctagccagtatggtgcaccggcgcaaaccccgtctagccagtatggtgcaccggcgcaaaccccgtctagccagtatggtgcaccggcgccctgaatactgcagatatttaggtgagaattcgc

SEQ ID NO: 29 - protein, 94 aa, Synthetic
HMHHHHHHASVLVFRAQTPSSQYGAPAQTPSSQYGAPAQTPSSQYGAPAQTPSSQYGAPAQTPSSQYGAPAQTPSSQYGAPAQTPSSQYGAPAP

SEQ ID NO: 30 - DNA, 415 bp, Synthetic
gccatatgcatcaccatcaccatcacgccagtgtgctggtattcagggcgcaaaccccgtctagccagtatggtgcaccggcgcaaaccccgtctagccagtatggtgcaccggcgcaaaccccgtctagccagtatggtgcaccggcgcaaaccccgtctagccagtatggtgcaccggcgcaaaccccgtctagccagtatggtgtaccggcgcaaaccccgtctagccagtatggtgcaccggcgcaaaccccgtctagccagtatggtgcaccggcgcaaaccccgtctagccagtatggtgcaccggcgcaaaccccgtctagccagtatggtgcaccggcgcaaaccccgtctagccagtatggtgcaccggcgccctgaatactgcagatatttaggtgagaattcgc

SEQ ID NO: 31 - protein, 127 aa, Synthetic
HMHHHHHHASVLVFRAQTPSSQYGAPAQTPSSQYGAPAQTPSSQYGAPAQTPSSQYGAPAQTPSSQYGVPAQTPSSQYGAPAQTPSSQYGAPAQTPSSQYGAPAQTPSSQYGAPAQTPSSQYGAPAP

SEQ ID NO: 32 - protein, 26 aa, Synthetic
RPSDTYGAPGGGNGGRPSDTYGAPGG